SEQUENCE LISTING (21 sequences)

SEQ ID NO: 1 - protein, 23 aa, synthetic construct
SACLQSLRTQLLTWALVGDVGQP

SEQ ID NO: 2 - protein, 16 aa, synthetic construct
AGISRELVDKLAAALE

SEQ ID NO: 3 - protein, 5 aa, synthetic construct
RKRRQ

SEQ ID NO: 4 - protein, 8 aa, synthetic construct
SDSCPHRP

SEQ ID NO: 5 - protein, 21 aa, synthetic construct
SKNLYSFYTEASIELHLNSHS

SEQ ID NO: 6 - protein, 12 aa, synthetic construct
SSLGCCECKSVR

SEQ ID NO: 7 - protein, 8 aa, synthetic construct
SEKHPHRP

SEQ ID NO: 8 - protein, 39 aa, synthetic construct
TDSTPALLSATVTPQKAKLGDTKELEAFIADLDKTLASM

SEQ ID NO: 9 - protein, 17 aa, synthetic construct
SSERNGQFPWPLKMFLT

SEQ ID NO: 10 - protein, 7 aa, synthetic construct
KFFQNLS

SEQ ID NO: 11 - protein, 10 aa, synthetic construct
INTDSIKLIA

SEQ ID NO: 12 - protein, 9 aa, synthetic construct
SKNLYSFLY

SEQ ID NO: 13 - protein, 8 aa, synthetic construct
SVDCRTCC

SEQ ID NO: 14 - protein, 70 aa, synthetic construct
SNEANRFSFILVLRGCYNFLFLWSLEGSCLIERKETNRKFYDIRAYDILFGDTPRPAQAEDLYEILDSLY

SEQ ID NO: 15 - protein, 25 aa, synthetic construct
DEIFTLKLIEGGALGKCEVMRVEPS

SEQ ID NO: 16 - protein, 43 aa, synthetic construct
SVAVSQDCTTALHPGQQSETLSQKKKGLQRXRQDYFFXLNLFF

SEQ ID NO: 17 - protein, 18 aa, synthetic construct
GKYNSTFTSSIIHNKNMK

SEQ ID NO: 18 - protein, 25 aa, synthetic construct
SGSLEVRSCTPAWVTERNFISKKKG

SEQ ID NO: 19 - DNA, 21 bp, synthetic construct
  source 1..21: mol_type = other DNA
gttctatccgcaacgttatgg

SEQ ID NO: 20 - DNA, 22 bp, synthetic construct
  source 1..22: mol_type = other DNA
ggaggaaagtcgttttttgggg

SEQ ID NO: 21 - DNA, 21 bp, synthetic construct
  source 1..21: mol_type = other DNA
tgctaaggacaacgttatcgg

(19) United States
(12) Patent Application Publication
(10) Pub. No.: US 2023/0341415 A1
(43) Pub. Date: Oct. 26, 2023

(54) MARKERS TO DISCRIMINATE SARCOIDOSIS FROM HEALTHY CONTROLS, TUBERCULOSIS AND LUNG CANCERS

(71) Applicant: Wayne State University, Detroit, MI (US)

(72) Inventor: Lobelia Samavati, Beverly Hills, MI (US)

(73) Assignee: Wayne State University, Detroit, MI (US)

(21) Appl. No.: 18/046,460

(22) Filed: Oct. 13, 2022

Related U.S. Application Data
(60) Provisional application No. 63/255,932, filed on Oct. 14, 2021.

(51) Int. Cl. G01N 33/68; C12Q 1/6883

(52) U.S. Cl. CPC G01N 33/6893; C12Q 1/6883; G01N 2800/7095; G01N 2458/00

(57) ABSTRACT

Systems, kits, methods to diagnose sarcoidosis are described. In addition to diagnosing sarcoidosis, the systems and methods can distinguish sarcoidosis from tuberculosis and lung cancer.

### CROSS-REFERENCE TO RELATED APPLICATION

This application claims priority to and the benefit of the earlier filing of U.S. Provisional Application No. 63/255,932, filed on Oct. 14, 2021, which is incorporated by reference herein in its entirety.

### STATEMENT OF GOVERNMENT INTEREST

This invention was made with government support under grant HL104481 awarded by the National Institutes of Health. The government has certain rights in the invention.

### INCORPORATION BY REFERENCE OF SEQUENCE LISTING

A computer readable XML file, entitled “W063-0083_SeqList.xml” created on or about Oct. 10, 2022, with a file size of 20 KB, contains the sequence listing for this application and is hereby incorporated by reference in its entirety.

### FIELD OF THE DISCLOSURE

The current disclosure provides systems and methods to diagnose sarcoidosis. In addition to diagnosing sarcoidosis, the systems and methods can distinguish sarcoidosis from tuberculosis. Further disclosed is a cDNA library and methods of its use for reliably identifying sarcoidosis markers.

### BACKGROUND OF THE DISCLOSURE

Sarcoidosis, also called sarcoid, is a disease involving abnormal collections of inflammatory cells (granulomas) that can form as nodules in multiple organs. The granulomas are most often located in the lungs or its associated lymph nodes. The disease seems to be caused by an immune reaction to an infection or some other trigger.

Diagnosis of sarcoidosis is challenging as the signs and symptoms of the condition are very broad, sometimes mimicking symptoms of other diseases. Further, symptoms can vary widely according to the organ system affected by the disorder. This variance can lead to a delay in diagnosis, or inappropriate treatment, therefore demonstrating a need for improved sarcoidosis diagnostic techniques.

The symptoms of sarcoidosis can also particularly resemble those caused by infection with tuberculosis. Thus, ability of a diagnostic to reliably distinguish between sarcoidosis and tuberculosis infection would allow faster treatment of each condition, resulting in better treatment outcomes.

### SUMMARY OF THE DISCLOSURE

The present disclosure provides systems and methods to diagnose sarcoidosis in a subject. The systems and methods can distinguish a sarcoidosis subject from a healthy subject and/or a subject having tuberculosis. The systems and methods include diagnostic kits. The systems and methods also include a cDNA library to identify markers for sarcoidosis or tuberculosis diagnosis as well as methods of using the cDNA library to identify such markers, among others.

A first embodiment is a method of diagnosing sarcoidosis in a subject, the method including assaying a sample derived from a subject for the presence of one or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and diagnosing the subject as healthy or having sarcoidosis based on the up- or down-regulation of the one or more markers, as compared to a reference level for each marker. In examples of this embodiment, the method includes assaying the sample for the presence of CFL1, 4FLI_A, ITPR3, CCL22, DSP, RAB36, PAR4, RGC32, DPY19L2, ERBB4, NEXMIF, or 1ZZP; and diagnosing the subject as healthy or having sarcoidosis based on the up- or down-regulation of the one or more markers. In further examples, the method includes assaying the sample for the presence of IL17A, SH3YL1, RAB12, TRG10, POLKB, or INADL; and diagnosing the subject as healthy or having sarcoidosis based on the up- or down-regulation of the one or more markers. In yet more examples, the method includes assaying the sample for the presence of two, three, four, five, six, seven, eight, nine, ten, eleven, twelve, thirteen, or more of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and diagnosing the subject as healthy or having sarcoidosis based on the up- or down-regulation of the one or more markers.

In any of these methods, the method may further include assaying the sample for the presence of at least one of CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; and diagnosing the subject as healthy or having sarcoidosis based on the up- or down-regulation of the one or more markers.

Also provided is a kit for diagnosing sarcoidosis in a subject, wherein the kit includes a protein that binds one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; and a detectable label. Examples of such kits include one or more proteins that bind one of CFL1, 4FLI_A, ITPR3, CCL22, DSP, RAB36, PAR4, RGC32, DPY19L2, ERBB4, NEXMIF, or 1ZZP; and a detectable label. Additional examples of the kits include one or more proteins that bind IL7A, SH3YL1, RAB12, TRG10, POLKB, or INADL; and a detectable label. Yet more examples kits include two, three, four, five, six, seven, eight, nine, ten, eleven, twelve, thirteen, or more proteins that each one of bind of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL, and a detectable label. Further examples of the kit further include one or more proteins that bind CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; and a detectable label. In any of these kits, the proteins may include antibodies, epitopes or mimotopes.

Also provided is a kit embodiment for diagnosing sarcoidosis in a subject wherein the kit includes a nucleic acid that binds a gene encoding CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; and a detectable label. In examples of this kit embodiment, there are provided kits that include one or more nucleic acids that bind a gene encoding CFL1, 4FLI_A, ITPR3, CCL22, DSP, RAB36, PAR4, RGC32, DPY19L2, ERBB4, NEXMIF, or 1ZZP; and a detectable label. Additional example kits include one or more nucleic acids that bind a gene encoding IL7A, SH3YL1, RAB12, TRG10, POLKB, or INADL; and a detectable label. Yet further example kits include two, three, four, five, six, seven, eight, nine, ten, eleven, twelve, thirteen, or more nucleic acids each of which binds a gene encoding one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and a detectable label. Additional example kits further include one or more nucleic acids that bind a gene encoding CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv01890; BfrA; DAB2; or TCEB2; and a detectable label. In any of the kits of these embodiment, the detectable label may be a radioactive isotope, enzyme, dye, fluorescent dye, magnetic bead, or biotin.

In any of the kit embodiments, optionally the kit may further include reagents to perform an enzyme-linked immunosorbent assay (ELISA), a radioimmunoassay (RIA), a Western blot, an immunoprecipitation, an immunohistochemical staining, flow cytometry, fluorescence-activated cell sorting (FACS), an enzyme substrate color method, and/or an antigen-antibody agglutination.

Yet another embodiment is a method of diagnosing sarcoidosis in a subject, the method including: obtaining a sample from a subject; assaying the sample for one or more markers selected from CFL1, 4FLI_A, ITPR3, CCL22, DSP, RAB36, PAR4, RGC32, DPY19L2, ERBB4, NEXMIF, 1ZZP, IL7A, SH3YL1, RAB12, TRG10, POLKB, and INADL; obtaining a value based on the assay; comparing the value to a reference level; and diagnosing the subject as healthy or having sarcoidosis based on the up- or down-regulation of the one or more markers as demonstrated by the value and the reference level. By way of example, such methods may include assaying the sample for one or more markers selected from CFL1, 4FLI_A, ITPR3, CCL22, DSP, RAB36, PAR4, RGC32, DPY19L2, ERBB4, NEXMIF, and 1ZZP. In additional examples of this method embodiment, the method includes assaying the sample for one or more markers selected from IL17A, SH3YL1, RAB12, TRG10, POLKB, and INADL. For instance, the method may include assaying the sample for two, three, four, five, six, seven, eight, nine, ten, eleven, twelve, thirteen, or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL. Examples of the method further include assaying the sample for one or more markers selected from CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2.

In any of the provided methods, example methods include assaying the sample for one or more markers include contacting the sample with a probe including a detectable label, wherein the probe binds the marker. In any of the provided methods, example methods include obtaining a value based on the assay includes analyzing the binding of the probe to the marker in the sample. In any of the provided methods, example methods include analyzing the binding of the probe to the marker in the sample includes quantitating the amount of the marker in the sample. In any of the provided methods, example methods include the sample is a tissue sample, a cell sample, a whole blood sample, a serum sample, a plasma sample, a saliva sample, a sputum sample, or a urine sample. In some examples of the method, the value is a score, such as a weighted score.

Another provided embodiment is a microarray including one or more proteins each of which binds one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL.

An additional embodiment is a microarray including one or more proteins each of which binds one of CFL1, 4FLI_A, ITPR3, CCL22, DSP, RAB36, PAR4, RGC32, DPY19L2, ERBB4, NEXMIF, or 1ZZP.

Yet another embodiment is a microarray including one or more proteins each of which binds one of IL17A, SH3YL1, RAB12, TRG10, POLKB, or INADL.

In any of the three preceding embodiments, the microarray optionally may further include one or more proteins each of which binds one of CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2.

Another embodiment is a microarray including a nucleic acid that binds to a gene encoding CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL.

Also provided is a microarray embodiment, including a nucleic acid that binds a gene encoding CFL1, 4FLI_A, ITPR3, CCL22, DSP, RAB36, PAR4, RGC32, DPY19L2, ERBB4, NEXMIF, or 1ZZP.

Yet another embodiment is a microarray including a nucleic acid that binds a gene encoding: IL17A, SH3YL1, RAB12, TRG10, POLKB, or INADL.

In any of the three preceding embodiments, the microarray optionally may further include at least one nucleic acid that binds a gene encoding CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv01890; BfrA; DAB2; or TCEB2.

Another provided embodiment is a microarray including one or more of the following proteins or an identifying peptide therefrom CFL1, 4FLI_A, ITPR3, CCL22, DSP, RAB36, PAR4, RGC32, DPY19L2, ERBB4, NEXMIF, 1ZZP, IL7A, SH3YL1, RAB12, TRG10, POLKB, or INADL.

Also provided is a microarray embodiment including one or more of the following proteins or an identifying peptide therefrom CFL1, 4FLI_A, ITPR3, CCL22, DSP, RAB36, PAR4, RGC32, DPY19L2, ERBB4, NEXMIF, or 1ZZP.

Another provided microarray embodiment includes one or more of the following proteins or an identifying peptide therefrom: IL17A, SH3YL1, RAB12, TRG10, POLKB, or INADL.

In any of the three preceding embodiments, the microarray optionally may further include one or more of the following proteins or a identifying peptide therefrom: CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv01890; BfrA; DAB2; or TCEB2.

In any of the described microarrays, the protein or the nucleic acid on the microarray may optionally include a label that can be detected.

In any of the described microarrays, the microarray optionally may include two or more, three or more, four or more, five or more, six or more, seven or more, eight or more, or nine or more of the proteins (or nucleic acids) on the microarray.

In any of the described microarray embodiments, examples also include microarrays that include two or more, three or more, four or more, five or more, six or more, seven or more, eight or more, or nine or more of the nucleic acids on the microarray.

Also provide is an embodiment of a kit including at least one microarray of any one of the other embodiments described herein. Optionally, such kits may utilize at least one clone or marker sequence identified herein, and wherein the kit comprises reagents to perform an enzyme-linked immunosorbent assay (ELISA), to detect specific immunoglobulin (IgG, IgA and Ig M).

Yet another embodiment is a method of serological diagnosis of sarcoidosis, and/or a method of distinguishing sarcoidosis from other granulomatous diseases (such as tuberculosis), comprising detecting one or more immunoglobulin (e.g., IgG, IgA and Ig M) specific for and/or immunoreactive to at least one clone or marker sequence identified herein.

### BRIEF DESCRIPTION OF THE FIGURES

This application contains at least one drawing executed in color. Copies of this application with color drawing(s) will be provided by the Office upon request and payment of the necessary fee.

FIGS. 1A-1F. PCA and Hierarchal clustering (option 1). (FIG. 1A) PCA plot along PC1 and PC2 generated with 1070 clones of four groups: (1) healthy control samples (black circles); (2) Sarcoidosis samples (red squares); (3) TB samples (blue diamond); and (4) Lung cancer (blue triangle). Biomarker clusters along the PC1 explain a variance of only 14%, while the variance along PC2 was about 13%. (FIG. 1B) The hierarchal clustering was applied on the healthy controls (black labels), sarcoidosis (red labels), TB patients (blue labels) and lung cancer (blue labels) with 1070 clones. (FIG. 1C) PCA plot along the PC1 and PC2 results when applied on 132 sarcoidosis clones. The PC1 explained 0.33 of variance, whereas PC2 explained 13% of the variance. As shown, the sarcoidosis samples are well separated from the lung cancer, TB controls and most healthy control samples. (FIG. 1D) Hierarchal clustering using only the top 132 sarcoidosis clones (FDR 0.05). (FIG. 1E) PCA plot generated with the top 14 sarcoidosis clones. The PC1 explained 45% of the variance, whereas PC2 explained 16% of the variance. (FIG. 1F) Hierarchal clustering using the top 14 sarcoidosis clones. This figure demonstrates better clustering with the top 14 sarcoidosis clones and the 132 significant sarcoidosis clones (panels FIGS. 1C, 1D, 1E, and 1F) when compared to the clustering using all clones (panels FIG. 1A and FIG. 1B).

FIGS. 2A-2D. PCA and Hierarchal clustering (option 2). (FIG. 2A) PCA plot along PC1 and PC2 generated with 221 clones (FDR 0.05) of the four groups: (1) healthy control samples (black circles); (2) Sarcoidosis samples (red squares); (3) TB samples (blue diamond); and (4) Lung cancer (blue triangle). The PC1 explains a variance of 32%, while the variance along PC2 was 12%. (FIG. 2B) The hierarchal clustering was applied on the healthy controls (black labels), sarcoidosis (red labels), TB patients (blue labels) and lung cancer (blue labels) with 221 clones (FDR 0.05). (FIG. 2C) PCA plot along the PC1 and PC2 results when applied on the top 12 sarcoidosis clones. The PC1 explained 54% of the variance, whereas PC2 explained 14% of the variance. As shown, the sarcoidosis samples are well separated from the lung cancer, TB controls and most healthy control samples. (FIG. 2D) Hierarchal clustering using the top 12 sarcoidosis clones. This figure demonstrates well clustering with top 12 sarcoidosis classifier clones.

FIGS. 3A and 3B. Diagrammatic Representation significant clones from two approaches (option 1 and 2). (FIG. 3A) Illustrates the Venn diagram of 132 clones (FDR 0.05) from option 1 and 221 clones (FDR 0.01) from option 2. (FIG. 3B) depicts the Venn diagram of the 14 classifiers clones from option 1 and 12 clones from option 2.

FIG. 4: Displays a heatmap plot of the distinct expression features of the final clones identified in option 1 and 2.

FIG. 5. Classification to predict sarcoidosis from healthy controls, TB patients and LC patients (the first row option 1 and the second row option 2). (FIG. 5A) Performance of 132 clones on the testing set. (FIG. 5B) Performance of the top 14 classifier clones on the test set. The ROC curves demonstrate excellent classification performance with AUC of 0.947 with sensitivity of 0.883 and specificity of 0.923. (FIG. 5C) Performance of 221 clones on the testing set. (FIG. 5D) Performance of the top 12 clones on the test set. The ROC curves demonstrate strong classification performance with AUC of 0.926 with sensitivity of 0.962 and specificity of 0.837.

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. In the Sequence Listing:

SEQ ID NOs: 1-18 are the amino acid sequences of mimotopes in-frame with T7 10B gene, as follows: SACLQSLRTQLLTWALVGDVGQP (SEQ ID NO: 1); AGISRELVDKLAAALE (SEQ ID NO: 2); RKRRQ (SEQ ID NO: 3); SDSCPHRP (SEQ ID NO: 4); SKNLYSFYTEASIELHLNSHS (SEQ ID NO: 5); SSLGCCECKSVR (SEQ ID NO: 6); SEKHPHRP (SEQ ID NO: 7); TDSTPALLSATVTPQKAKLGDTKELEAFIADLDKTLASM (SEQ ID NO: 8); SSERNGQFPWPLKMFLT (SEQ ID NO: 9); KFFQNLS (SEQ ID NO: 10); INTDSIKLIA (SEQ ID NO: 11); SKNLYSFLY (SEQ ID NO: 12); SVDCRTCC (SEQ ID NO: 13); SNEANRFSFILVLRGCYNFLFLWSLEGSCLIERKETNRKFYDIRAYDILFGDTPRPAQAEDLYEIL DSLY (SEQ ID NO: 14); DEIFTLKLIEGGALGKCEVMRVEPS (SEQ ID NO: 15); SVAVSQDCTTALHPGQQSETLSQKKKGLQRXRQDYFFXLNLFF (SEQ ID NO: 16); GKYNSTFTSSIIHNKNMK (SEQ ID NO: 17); and SGSLEVRSCTPAWVTERNFISKKKG (SEQ ID NO: 18). See also Table 7 for additional information.

SEQ ID NOs: 19-21 are the nucleic acid sequences of the T7 phage forward primer GTTCTATCCGCAACGTTATGG (SEQ ID NO: 19); the T7 phage reverse primer GGAGGAAAGTCGTTTTTTGGGG (SEQ ID NO: 20); and the T7 phage sequence primer TGCTAAGGACAACGTTATCGG (SEQ ID NO: 21).

### DETAILED DESCRIPTION

Sarcoidosis is a multisystem granulomatous inflammatory disease. The disease is typically characterized by the formation of small, granular inflammatory lesions or granulomas (e.g., non-caseating granulomas) in a variety of organs, and/or the presence of immune responses (e.g., presence of CD4+ T lymphocytes and macrophages) in affected tissues or organs. Granulomatous inflammation may be attributed to the accumulation of monocytes, macrophages, and a pronounced Th1 response and activated T-lymphocytes, with elevated production of TNFα, IL-2, IL-12, IFNγ, IL-1, IL-6 or IL-15.

Exemplary subtypes of sarcoidosis include systemic sarcoidosis, Lofgren's syndrome, pulmonary sarcoidosis, cutaneous sarcoidosis, neurosarcoidosis, cardiac sarcoidosis, ocular sarcoidosis, hepatic sarcoidosis, musculoskeletal sarcoidosis, renal sarcoidosis, or sarcoidosis with the involvement of other organs or tissues.

Systemic sarcoidosis is sarcoidosis with multiple organ involvement. Symptoms of systemic sarcoidosis include aches, arthritis, chills, dry mouth, enlarged lymph glands (e.g., armpit lump), fatigue, fever, loss of appetite, night sweats, nosebleed, pains, persistent cough, malaise, shortness of breath, weakness, and weight loss. Because systemic sarcoidosis involves multiple organs, symptoms described below for other more particular types of sarcoidosis can also be relevant to systemic sarcoidosis.

Lofgren's syndrome represents an acute presentation of systemic sarcoidosis, typically characterized by the triad of erythema nodosum, bilateral hilar adenopathy and arthritis or arthralgias. It can also be accompanied by fever.

Pulmonary sarcoidosis refers to sarcoidosis that affects pulmonary tissues or organs (e.g., lungs). Symptoms of pulmonary sarcoidosis usually include normal, abnormal or deteriorating lung function; abnormal lung stiffness; bleeding from the lung tissue; cough; decreased lung volume; decreased vital capacity (full breath in, to full breath out); enlarged lymph nodes in the chest; granulomas in alveolar septa, bronchiolar, and/or bronchial walls; higher than normal expiratory flow ratios; an increased FEV₁/FVC ratio; limited amount of air drawn into the lungs; loss of lung volume; obstructive lung changes; pulmonary hypertension; pulmonary failure; scarring of lung tissue; and/or shortness of breath.

Cutaneous sarcoidosis is a complication of sarcoidosis with skin involvement. Cutaneous sarcoidosis includes annular sarcoidosis, erythrodermic sarcoidosis, hypopigmented sarcoidosis, ichthyosiform sarcoidosis, morpheaform sarcoidosis, mucosal sarcoidosis, papular sarcoid, scar sarcoid, subcutaneous sarcoidosis and ulcerative sarcoidosis. Symptoms of cutaneous sarcoidosis include erythema nodosum (e.g., raised, red, firm skin sores, cellulitis, furunculosis or other inflammatory panniculitis); hair loss; lupus pernio (e.g., scar or discoid lupus erythematosus); maculopapular eruptions; nodular lesions; papules (e.g., granulomatous rosacea, acne or benign appendageal tumors); skin lesions; skin plaques (e.g., psoriasis, lichen planus, nummular eczema, discoid lupus erythematosus, granuloma annulare, cutaneous T-cell lymphoma, Kaposi's sarcoma or secondary syphilis); skin rashes, and/or scars becoming more raised.

Neurosarcoidosis or neurosarcoid refers to sarcoidosis in which inflammation and abnormal deposits occur in the brain, spinal cord, and any other areas of the nervous system. Symptoms of neurosarcoidosis can include abnormal or loss of sense of smell; abnormal or loss of sense of taste; carpal tunnel syndrome; changes in menstrual periods; confusion; decreased hearing; delirium; dementia; disorientation; dizziness; double vision or other vision problems or changes; excessive thirst; excessive tiredness (e.g., fatigue); facial palsy, weakness or drooping; headache; high urine output; hypopituitarism; loss of bowel or bladder control; muscle weakness; paraplegia; psychiatric disturbances; radicular pain; retinopathy; seizures; sensory losses; speech impairment; and/or vertigo.

The systems and methods disclosed herein can be used to diagnose sarcoidosis. In particular embodiments, the diagnosed sarcoidosis is systemic sarcoidosis, pulmonary sarcoidosis, cutaneous sarcoidosis, Lofgren's syndrome, neurosarcoidosis, cardiac sarcoidosis, ocular sarcoidosis, hepatic sarcoidosis, musculoskeletal sarcoidosis, renal sarcoidosis, or sarcoidosis with the involvement of other organs or tissues. In more particular embodiments, the systems and methods disclosed herein can be used to diagnose pulmonary sarcoidosis, neurosarcoidosis, and/or ocular sarcoidosis.

Typically, a sarcoidosis patient will present with symptoms described above or clinical features set out in the Statement on Sarcoidosis published by the American Thoracic Society (Am. J. Respir. Crit. Care Med. 160(2):736-55, 1999). Sarcoidosis patients may often, however, be asymptomatic. Further the common symptoms of sarcoidosis are vague, and can sometimes be similar to symptoms of numerous other conditions including lymphoma and tuberculosis. Thus, diagnosis is difficult.

Currently, subjects with suspected sarcoidosis are typically assessed with a chest assessment for pulmonary involvement, as the vast majority of sarcoidosis subjects have pulmonary involvement. These assessments are generally based upon a bronchoscopy with biopsy; chest X-ray; CT scan; CT-guided biopsy; lung gallium (Ga) scan; mediastinoscopy; open lung biopsy; PET scan and/or a radiograph. Radiographs are typically assigned a stage of 0-4 according to the presence or absence of hilar adenopathy and parenchymal disease. Thus there are five stages: Stage 0: no visible intrathoracic findings; Stage 1: bilateral hilar lymphadenopathy (BHL), which may be accompanied by paratracheal adenopathy/lung fields are clear of infiltrates; Stage 2: bilateral hilar adenopathy (BHL) accompanied by parenchymal infiltration; Stage 3: parenchymal infiltration without bilateral hilar adenopathy (BHL); or Stage 4: advanced pulmonary fibrosis with evidence of honey-combing, hilar retraction, bullae, cysts, and emphysema.

The present disclosure provides significant advancements in the diagnosis of sarcoidosis because diagnosis can be achieved with, for example, a blood test and can distinguish sarcoidosis subjects from healthy subjects and/or subjects having tuberculosis.

The systems and methods disclosed herein were achieved by creating and screening a complex cDNA library. Particularly, a heterologous cDNA library derived from bronchoalveolar cell (BAL) samples and total white blood cells (WBC) from sarcoidosis patients was developed. Both sarcoid-derived libraries were combined with cultured human monocytes and embryonic lung fibroblast cDNA libraries to build a complex sarcoidosis library (CSL). Differential biopanning for negative and positive selection was performed using sera from healthy controls to remove non-specific IgG, and sarcoidosis sera for selective enrichment. Four rounds of biopannings were performed and the selected phage libraries were used for microarray immunoscreening. Each cycle of biopanning included passing the entire phage library through protein G beads coated with IgG from pooled sera of healthy controls, then passing through beads coated with IgGs from individual serum of sarcoid subjects.

After biopanning, phage clones were randomly selected and amplified and their lysates were arrayed in quintuplicates onto slides (Grace Biolabs, OR) using the ProSys 5510TL robot (Cartesian Technologies, CA). It was tested whether this novel library representing relevant antigens would specifically recognize high IgG titer in sera of sarcoidosis subjects.

Using bioinformatics tools, a large number of markers with high sensitivity and specificity were identified that discriminate among the sera of patients with sarcoidosis, healthy controls and TB. Using the integrative-analysis method that combines results from two independent trials, clones that significantly differentiated sarcoidosis from controls were identified. Similarly, clones that differentially reacted with TB sera and not with sarcoidosis or control sera were identified. Furthermore, the top 10 discriminating antigens for TB and sarcoidosis were sequenced and homologies were identified in a public data base. These data indicate development of a unique library enabling the detection of highly significant antigens to discriminate between patients with sarcoidosis and tuberculosis.

An antigen is a substance that induces an immune response. Accordingly, the antigens detected from the library are markers useful for diagnosing sarcoidosis and TB.

The systems and methods diagnose sarcoidosis by assaying a sample obtained from a subject for the up- or down-regulation of one or more markers associated with sarcoidosis. Previously recognized markers include Small inducible cytokine A21 precursor (CCL21); Methionine aminopeptidase 1 (Metap1); Activated RNA polymerase II transcription cofactor variant 4 (PC4); RNA methyltransferase (CLI_3190); Tumor necrosis factor receptor superfamily member 21 precursor (also known as death receptor 6 (DR6)) (TNFRSF21); Monocyte differentiation antigen CD14 (CD14); DnaJ (Hsp40) homolog subfamily C member 1 precursor (DNAJC1); Amyloid β A4 precursor protein-binding family B member 1-interacting protein (APBB1); Fibroblast growth factor binding protein 2 precursor (FGFBP-2); SH3 domain-containing YSC84 like protein 1 (SH3YL1); thioester reductase [Pseudomonas fluorescens] (PFWH6_0117); histidine kinase [Pseudomonas fluorescens] (PFL_3193); Homo sapiens chromatin modifying protein 4B (CHMP4B); hypothetical protein [Porphyromonas somerae] Peptidase family C39 mostly contains bacteriocin-processing endopeptidases from bacteria; truncated HIC1 protein [Homo sapiens] (H1C1); replication protein [Mycobacterium] (MVAC_06252); Homo sapiens ribosomal protein S2 (RPS2); triosephosphate isomerase [Mycobacterium tuberculosis] (tpiA); membrane protein [Mycobacterium tuberculosis] (Rv2563); serine/threonine protein kinase [Mycobacterium tuberculosis] (Rv0410C); PPE family protein [Mycobacterium tuberculosis RGTB423] (MRGA423_16320); rRNA methyltransferase [Mycobacterium tuberculosis] (Rv0881); peroxisome biogenesis factor 10 isoform 1 [Homo sapiens] (PEX10); sulfate ABC transporter permease [Mycobacterium tuberculosis] (CysU); and/or D-alpha-D-heptose-7-phosphate kinase [Mycobacterium tuberculosis] (hddA). Additional markers of sarcoidosis are described in Example 2, as well as in Appendix I submitted herewith.

In particular embodiments, the systems and methods diagnose sarcoidosis by assaying a sample obtained from a subject for the up- or down-regulation of two or more; three or more; four or more; five or more; six or more; seven or more; eight or more; nine or more or ten or more markers associated with sarcoidosis disclosed herein. In further embodiments, the systems and methods diagnose sarcoidosis by assaying a sample obtained from a subject for the up- or down-regulation of two; three; four; five; six; seven; eight; nine or ten markers associated with sarcoidosis disclosed herein.

In one embodiment, the markers include (referred to by gene abbreviations for brevity) on or more of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL. In another embodiment, the markers include one or more of CFL1, 4FLI_A, ITPR3, CCL22, DSP, RAB36, PAR4, RGC32, DPY19L2, ERBB4, NEXMIF, and 1ZZP. In another embodiment, the markers include one or more of IL17A, SH3YL1, RAB12, TRG10, POLKB, and INADL. In another embodiment, the markers further include at least one of CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2. selected from; and diagnosing the subject as healthy or having sarcoidosis based on the up- or down-regulation of the one or more markers, as compared to a reference level for each marker.

In particular embodiments, the systems and methods distinguish sarcoidosis from tuberculosis in a subject by assaying a sample obtained from a subject for the up- or down-regulation of two or more; three or more; four or more; five or more; six or more; seven or more; eight or more; nine or more or ten or more markers that distinguish sarcoidosis from tuberculosis disclosed herein. In further embodiments, the systems and methods distinguish sarcoidosis from tuberculosis by assaying a sample obtained from a subject for the up- or down-regulation of two; three; four; five; six; seven; eight; nine or ten markers associated with sarcoidosis disclosed herein.

“Up-regulation” or “up-regulated” means an increase in the presence of a protein and/or an increase in the expression of its gene. “Down-regulation” or “down-regulated” means a decrease in the presence of a protein and/or a decrease in the expression of its gene. “It's gene” in reference to a particular protein refers to a nucleic acid sequence (used interchangeably with polynucleotide or nucleotide sequence) that encodes the particular protein. This definition also includes various sequence polymorphisms, mutations, and/or sequence variants wherein such alterations do not substantially affect the identity or function of the particular protein. For example, in a sequence identity analysis, the test protein would share at least 80% sequence identity; at least 81% sequence identity; at least 82% sequence identity; at least 83% sequence identity; at least 84% sequence identity; at least 85% sequence identity; at least 86% sequence identity; at least 87% sequence identity; at least 88% sequence identity; at least 89% sequence identity; at least 90% sequence identity; at least 91% sequence identity; at least 92% sequence identity; at least 93% sequence identity; at least 94% sequence identity; at least 95% sequence identity; at least 96% sequence identity; at least 97% sequence identity; at least 98% sequence identity or at least 99% sequence identity with the particular protein.

“% sequence identity” refers to a relationship between two or more sequences, as determined by comparing the sequences. In the art, “identity” also means the degree of sequence relatedness between protein (or nucleic acid) sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” mean any set of values or parameters which originally load with the software when first initialized.

The function of a protein can be assayed by a relevant activity assay. Function is not substantially affected if there is no statistically significant difference in activity between the particular protein and the test protein. Exemplary activity assays include binding assays, or, if the protein is an enzyme, enzyme activity assays including, for example, protease assays, kinase assays, phosphatase assays, reductase assays, etc. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

The term “gene” can include not only coding sequences but also regulatory regions such as promoters, enhancers, and termination regions. The term further can include all introns and other DNA sequences spliced from the mRNA transcript, along with variants resulting from alternative splice sites. Gene sequences encoding the particular protein can be DNA or RNA that directs the expression of the particular protein. These nucleic acid sequences may be a DNA strand sequence that is transcribed into RNA or an RNA sequence that is translated into the particular protein. The nucleic acid sequences include both the full-length nucleic acid sequences as well as non-full-length sequences derived from the full-length protein. The sequences can also include degenerate codons of the native sequence. Portions of complete gene sequences are referenced throughout the disclosure as is understood by one of ordinary skill in the art.

Up- or down-regulation of the markers, as indicated elsewhere herein for particular markers can be assessed by comparing a value to a relevant reference level. For example, the quantity of one or more markers can be indicated as a value. The value can be one or more numerical values resulting from the assaying of a sample, and can be derived, e.g., by measuring level(s) of the marker(s) in the sample by an assay performed in a laboratory, or from a dataset obtained from a provider such as a laboratory, or from a dataset stored on a server. The markers disclosed herein can be a protein marker or a nucleic acid marker (gene encoding the protein marker).

In the broadest sense, the value may be qualitative or quantitative. As such, where detection is qualitative, the systems and methods provide a reading or evaluation, e.g., assessment, of whether or not the marker is present in the sample being assayed. In yet other embodiments, the systems and methods provide a quantitative detection of whether the marker is present in the sample being assayed, i.e., an evaluation or assessment of the actual amount or relative abundance of the marker in the sample being assayed. In such embodiments, the quantitative detection may be absolute or, if the method is a method of detecting two or more different markers in a sample, relative. As such, the term “quantifying” when used in the context of quantifying a marker in a sample can refer to absolute or to relative quantification. Absolute quantification can be accomplished by inclusion of known concentration(s) of one or more control markers and referencing, e.g., normalizing, the detected level of the marker with the known control markers (e.g., through generation of a standard curve). Alternatively, relative quantification can be accomplished by comparison of detected levels or amounts between two or more different markers to provide a relative quantification of each of the two or more markers, e.g., relative to each other. The actual measurement of values of the markers can be determined at the protein or nucleic acid level using any method known in the art. In some embodiments, a marker is detected by contacting a sample with reagents (e.g., antibodies or nucleic acid primers), generating complexes of reagent and marker(s), and detecting the complexes.

The reagent can include a probe. A probe is a molecule that binds a target, either directly or indirectly. The target can be a marker, a fragment of the marker, or any molecule that is to be detected. In embodiments, the probe includes a nucleic acid or a protein. As an example, a protein probe can be an antibody. An antibody can be a whole antibody or a fragment of an antibody, A probe can be labeled with a detectable label. Examples of detectable labels include fluorescers, chemiluminescers, dyes, enzymes, enzyme substrates, enzyme cofactors, enzyme inhibitors, enzyme subunits, metal ions, and radioactive isotopes.

“Protein” detection includes detection of full-length proteins, mature proteins, pre-proteins, polypeptides, isoforms, mutations, post-translationally modified proteins and variants thereof, and can be detected in any suitable manner.

Those skilled in the art will be familiar with numerous specific immunoassay formats and variations thereof which can be useful for carrying out the methods disclosed herein. See, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies can be conjugated to detectable labels or groups such as radiolabels (e.g., 35S, 125I, 131I), enzyme labels (e.g., horseradish peroxidase, alkaline phosphatase), and fluorescent labels (e.g., fluorescein, Alexa, green fluorescent protein, rhodamine) in accordance with known techniques.

Examples of suitable immunoassays include immunoblotting, immunoprecipitation, immunofluorescence, chemiluminescence, electro-chemiluminescence (ECL), and/or enzyme-linked immunoassays (ELISA).

Antibodies may also be useful for detecting post-translational modifications of markers. Examples of post-translational modifications include tyrosine phosphorylation, threonine phosphorylation, serine phosphorylation, citrullination and glycosylation (e.g., O-GlcNAc). Such antibodies specifically detect the phosphorylated amino acids in marker proteins of interest. These antibodies are well-known to those skilled in the art, and commercially available. Post-translational modifications can also be determined using metastable ions in reflector matrix-assisted laser desorption ionization-time of flight mass spectrometry (MALDI-TOF). See U. Wirth et al., Proteomics 2002, 2(10):1445-1451.

Up- or down-regulation of genes also can be detected using, for example, cDNA arrays, cDNA fragment fingerprinting, cDNA sequencing, clone hybridization, differential display, differential screening, FRET detection, liquid microarrays, PCR, RT-PCR, quantitative real-time RT-PCR analysis with TaqMan assays, molecular beacons, microelectric arrays, oligonucleotide arrays, polynucleotide arrays, serial analysis of gene expression (SAGE), and/or subtractive hybridization.

As an example, Northern hybridization analysis using probes which specifically recognize one or more marker sequences can be used to determine gene expression. Alternatively, expression can be measured using RT-PCR; e.g., polynucleotide primers specific for the differentially expressed marker mRNA sequences reverse-transcribe the mRNA into DNA, which is then amplified in PCR and can be visualized and quantified. Marker RNA can also be quantified using, for example, other target amplification methods, such as transcription mediated amplification (TMA), strand displacement amplification (SDA), and nucleic acid sequence based amplification (NASBA), or signal amplification methods (e.g., bDNA), and the like. Ribonuclease protection assays can also be used, using probes that specifically recognize one or more marker mRNA sequences, to determine gene expression.

Further hybridization technologies that may be used are described in, for example, U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992 as well as WO 95/21265; WO 96/31622; WO 97/10365; WO 97/27317; EP 373 203; and EP 785 280.

Proteins and nucleic acids can be linked to chips, such as microarray chips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Microarray refers to a solid carrier or support that has a plurality of molecules bound to its surface at defined locations. The solid carrier or support can be made of any material. As an example, the material can be hard, such as metal, glass, plastic, silicon, ceramics, and textured and porous materials; or soft materials, such as gels, rubbers, polymers, and other non-rigid materials. The material can also be nylon membranes, epoxy-glass and borofluorate-glass. The solid carrier or support can be flat, but need not be and can include any type of shape such as spherical shapes (e.g., beads or microspheres). The solid carrier or support can have a flat surface as in slides and micro-titer plates having one or more wells.

Binding to proteins or nucleic acids on microarrays can be detected by scanning the microarray with a variety of laser or CCD-based scanners, and extracting features with software packages, for example, Imagene (Biodiscovery, Hawthorne, CA), Feature Extraction Software (Agilent), Scanalyze (Eisen, M. 1999. SCANALYZE User Manual; Stanford Univ., Stanford, Calif. Ver 2.32), or GenePix (Axon Instruments).

Embodiments disclosed herein can be used with high throughput screening (HTS). Typically, HTS refers to a format that performs at least about 100 assays, at least about 500 assays, at least about 1000 assays, at least about 5000 assays, at least about 10,000 assays, or more per day. When enumerating assays, either the number of samples or the number of protein or nucleic acid markers assayed can be considered.

Generally HTS methods involve a logical or physical array of either the subject samples, or the protein or nucleic acid markers, or both. Appropriate array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis.

HTS assays and screening systems are commercially available from, for example, Zymark Corp. (Hopkinton, MA); Air Technical Industries (Mentor, OH); Beckman Instruments, Inc. (Fullerton, CA); Precision Systems, Inc. (Natick, MA), etc. These systems typically automate entire procedures including all sample and reagent pipetting, liquid dispensing, timed incubations, and final readings of the microplate in detector(s) appropriate for the assay. These configurable systems provide HTS as well as a high degree of flexibility and customization. The manufacturers of such systems provide detailed protocols for the various methods of HTS.

As stated previously, obtained marker values can be compared to a reference level. Reference levels can be obtained from one or more relevant datasets. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a population. A “population” is any grouping of subjects or samples of like specified characteristics. The grouping could be according to, for example, clinical parameters, clinical assessments, therapeutic regimens, disease status, severity of condition, etc.

Subjects include humans, veterinary animals (dogs, cats, reptiles, birds, hamsters, etc.) livestock (horses, cattle, goats, pigs, chickens, etc.), research animals (monkeys, rats, mice, fish, etc.) and other animals, such as zoo animals (e.g., bears, giraffe, elephant, lemurs).

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than or equal to 0.05.

In one embodiment, values obtained about the markers and/or other dataset components can be subjected to an analytic process with chosen parameters. The parameters of the analytic process may be those disclosed herein or those derived using the guidelines described herein. The analytic process used to generate a result may be any type of process capable of providing a result useful for classifying a sample, for example, comparison of the obtained value with a reference level, a linear algorithm, a quadratic algorithm, a decision tree algorithm, or a voting algorithm. The analytic process may set a threshold for determining the probability that a sample belongs to a given class. The probability preferably is at least at least 60%, at least 70%, at least 80%, at least 90%, at least 95% or higher.

In embodiments, the relevant reference level for a particular marker is obtained based on the particular marker in control subjects. Control subjects are those that are healthy and do not have sarcoidosis or tuberculosis. As an example, the relevant reference level can be the quantity of the particular marker in the control subjects.

Particular embodiments disclosed herein include obtaining a sample from a subject suspected of having sarcoidosis; assaying the sample for up- or down-regulation of one or more markers disclosed herein; determining one or more marker values based on the assaying; comparing the one or more marker values to a reference level; diagnosing sarcoidosis in the subject according to the up- or down regulation of a marker, as described elsewhere herein.

Particular embodiments also include distinguishing sarcoidosis from tuberculosis in a subject by obtaining a sample from a subject suspected of having sarcoidosis; assaying the sample for up- or down-regulation of one or more markers disclosed herein; determining one or more marker values based on the assaying; comparing the one or more marker values to a reference level; diagnosing sarcoidosis or tuberculosis in the subject according to the up- or down regulation of a marker, as described elsewhere herein.

The sample can be any appropriate biological sample obtained from the subject, such as a blood sample, a serum sample, a saliva sample, a urine sample, bronchoalveolar lavage sample, etc. The sample also can be obtained from a biopsy of an affected tissue or organ, such as a lung biopsy, or lymph gland biopsy. The sample can include cells of affected tissue or organ.

A diagnosis according to the systems and methods disclosed herein can direct a treatment regimen. For example, a sarcoidosis diagnosis can direct treatment with a sarcoidosis treatment (e.g., lifestyle and behavioral interventions; corticosteroids; methotrexate or azathioprine; hydroxychloroquine or chloroquine; cyclophosphamide or chlorambucil; pentoxifylline and thalidomide; infliximab or adalimumab; colchicine; various nonsteroidal anti-inflammatory drugs (NSAIDs, e.g., ibuprofen or aspirin); organ transplantation). A tuberculosis diagnosis can direct treatment with a tuberculosis treatment (e.g., isoniazid (INH); rifampin (RIF); ethambutol (EMB); pyrazinamide (PZA)). A healthy diagnosis can direct further medical analysis if the subject's symptoms suggest further analysis is warranted. Administered treatments will be delivered in therapeutically effective amounts leading to an improvement or resolution of the treated condition, as assessed by a practicing physician, veterinarian or researcher.

The systems and methods disclosed herein include kits. Disclosed kits include materials and reagents necessary to assay a sample obtained from a subject for one or more markers disclosed herein. The materials and reagents can include those necessary to assay the markers disclosed herein according to any method described herein and/or known to one of ordinary skill in the art.

Particular embodiments include materials and reagents necessary to assay for up- or down-regulation of a marker protein in a sample. In particular embodiments, the kits include antibodies to marker proteins and/or can also include aptamers, epitopes or mimotopes. Other embodiments additionally or alternatively include oligonucleotides that specifically assay for one or more marker nucleic acids based on homology and/or complementarity with marker nucleic acids. The oligonucleotide sequences may correspond to fragments of the marker nucleic acids. For example, the oligonucleotides can be more than 200, 175, 150, 100, 50, 25, 10, or fewer than 10 nucleotides in length. Collectively, any molecule (e.g., antibody, aptamer, epitope, mimotope, oligonucleotide) that forms a complex with a marker is referred to as a marker binding agent herein.

Embodiments of kits can contain in separate containers marker binding agents either bound to a matrix, or packaged separately with reagents for binding to a matrix. In particular embodiments, the matrix is, for example, a porous strip. In some embodiments, measurement or detection regions of the porous strip can include a plurality of sites containing marker binding agents. In some embodiments, the porous strip can also contain sites for negative and/or positive controls. Alternatively, control sites can be located on a separate strip from the porous strip. Optionally, the different detection sites can contain different amounts of marker binding agents, e.g., a higher amount in the first detection site and lesser amounts in subsequent sites. Upon the addition of test sample, the number of sites displaying a detectable signal provides a quantitative indication of the amount of marker present in the sample. The detection sites can be configured in any suitably detectable shape and can be, e.g., in the shape of a bar or dot spanning the width (or a portion thereof) of a porous strip.

In some embodiments the matrix can be a solid substrate, such as a “chip.” See, e.g., U.S. Pat. No. 5,744,305. In some embodiments the matrix can be a solution array; e.g., xMAP (Luminex, Austin, TX), Cyvera (Illumina, San Diego, CA), RayBio Antibody Arrays (RayBiotech, Inc., Norcross, GA), CellCard (Vitra Bioscience, Mountain View, CA) and Quantum Dots' Mosaic (Invitrogen, Carlsbad, CA).

Additional embodiments can include control formulations (positive and/or negative), and/or one or more detectable labels, such as fluorescein, green fluorescent protein, rhodamine, cyanine dyes, Alexa dyes, luciferase, and radiolabels, among others. Instructions for carrying out the assay, including, optionally, instructions for generating a score, can be included in the kit; e.g., written, tape, VCR, or CD-ROM.

In particular embodiments, the kits include materials and reagents necessary to conduct and immunoassay (e.g., ELISA). In particular embodiments, the kits include materials and reagents necessary to conduct hybridization assays (e.g., PCR). In particular embodiments, materials and reagents expressly exclude equipment (e.g., plate readers). In particular embodiments, kits can exclude materials and reagents commonly found in laboratory settings (pipettes; test tubes; distilled H₂O).

Numerous protein and gene sequence markers are disclosed herein. The disclosure is not limited to the particularly disclosed protein and gene sequences but instead also encompasses sequences including 80% sequence identity; 81% sequence identity; 82% sequence identity; 83% sequence identity; 84% sequence identity; 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity or 99% sequence identity.

When a protein sequence is provided, its gene sequences can be derived by one of ordinary skill in the art by, for example, consulting publicly available databases. In addition to the sequence identity parameters provided above, gene sequences that hybridize to derived sequences under high stringency conditions can also be included within the scope of the current disclosure. A gene or polynucleotide fragment “hybridizes” to another gene or polynucleotide fragment, such as a cDNA, genomic DNA, or RNA, when a single stranded form of the polynucleotide fragment anneals to the other polynucleotide fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Ed., Cold Spring Harbor Laboratory Press, Cold Spring Harbor (1989), particularly Chapter 11 and Table 11.1 therein (incorporated by reference herein for its teachings regarding the same). The conditions of temperature and ionic strength determine the “stringency” of the hybridization. Stringency conditions can be adjusted to screen for moderately similar fragments (such as homologous sequences from distantly related organisms) to highly similar fragments (such as genes that duplicate functional enzymes from closely related organisms). Post-hybridization washes determine stringency conditions. One set of hybridization conditions to demonstrate that sequences hybridize uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Stringent conditions use higher temperatures in which the washes are identical to those above except for the temperature of the final two 30 min washes in 0.2×SSC, 0.5% SDS is increased to 60° C. Highly stringent conditions use two final washes in 0.1SSC, 0.1% SDS at 65° C. Those of ordinary skill in the art will recognize that these temperature and wash solution salt concentrations may need to be adjusted as necessary according to factors such as the length of the hybridizing sequences.

Also disclosed herein is a cDNA library including mRNA isolated from (i) bronchoalveolar cells (BAL) of sarcoidosis patients; and (ii) white blood cells obtained from sarcoidosis patients. In further embodiments, the cDNA library further includes mRNA isolated from (iii) human splenic monocytes; and/or (iv) embryonic lung fibroblasts. The cDNA library can be screened for markers associated with sarcoidosis or related disorders. The cDNA library can be a phage display library, a ribosome display library, or a nucleic acid display library. In particular embodiments, the cDNA library is a T7 phage display library. In particular embodiments, the cDNA library should be biopanned to negatively select and/or enrich for detection markers of interest. For example, biopanning with samples from control subjects can remove potential hits that are non-specific to the condition of interest, resulting in negative selection. Biopanning with samples from subjects of interest (e.g., subjects having a condition of interest) selects potential hits that are specific to the condition of interest, resulting in enrichment of the cDNA library for hits of potential interest. The systems and methods disclosed herein include biopanning a cDNA library including mRNA isolated from (i) bronchoalveolar cells (BAL) of sarcoidosis patients; (ii) white blood cells obtained from sarcoidosis patients; (iii) human splenic monocytes; and (iv) embryonic lung fibroblasts to negatively select for and/or enrich the library for hits of interest.

In embodiments, the cDNA library is differentially biopanned to identify markers for sarcoidosis. As described above, differential biopanning involves biopanning by negative selection using sera from control subjects to remove non-specific IgG, followed by biopanning by positive enrichment using sera from sarcoidosis patients.

Additional embodiments include adhering cDNA expression products from a negatively selected and enriched cDNA library to a microarray. Additional embodiments include exposing the microarray to samples from subjects of interest and control samples. Additional embodiments include detecting cDNA expression products bound by molecules in samples from the subjects of interest. Additional embodiments include performing data analysis to identify molecules that bind cDNA expression products as markers of a condition of interest.

One embodiment includes detecting sarcoidosis or tuberculosis antigens by: (a) preparing a phage display library of sarcoidosis or tuberculosis antigens from cells of one or more subjects with sarcoidosis; (b) enriching the phage display library for sarcoidosis or tuberculosis antigens by biopanning; (c) selecting clones for amplification; (d) testing amplified clones for binding to antibodies in sera of sarcoidosis subjects; and (e) sequencing bound clones.

Another embodiment includes a library and method to identify sarcoidosis markers. One embodiment includes identifying proteins that bind to expression products of phage display clones derived from a library including mRNA isolated from (i) bronchoalveolar cells (BAL) of sarcoidosis patients; (ii) white blood cells obtained from sarcoidosis patients; (iii) human splenic monocytes; and/or (iv) embryonic lung fibroblasts. Another embodiment includes identifying proteins that bind to expression products of phage display clones derived from a library including mRNA isolated from (i) bronchoalveolar cells (BAL) of sarcoidosis patients; (ii) white blood cells obtained from sarcoidosis patients; (iii) human splenic monocytes; and (iv) embryonic lung fibroblasts. Following binding, identified proteins can be characterized and, in particular embodiments, synthesized.

These embodiments can be used to identify additional markers to diagnose systemic sarcoidosis, pulmonary sarcoidosis, cutaneous sarcoidosis, Lofgren's syndrome, neurosarcoidosis, cardiac sarcoidosis, ocular sarcoidosis, hepatic sarcoidosis, musculoskeletal sarcoidosis, renal sarcoidosis, or sarcoidosis with the involvement of other organs or tissues.

In embodiments, diagnosis of sarcoidosis may be achieved in accordance with the previously disclosed methods through the use of a computing device to provide for a quicker, more reliable, and less labor intensive diagnosis.

An illustrative schematic 1000 for diagnosing sarcoidosis in a subject 1002 on a computing device 1008, includes an illustrative diagram 1028 of a computing device 1008 implementing the diagnostic framework 1018. Sample biological material 1004 is collected from the subject 1002. That sample 1004 may be assayed for the presence of one or more markers. An indication of the up- or down-regulation of the markers is reflected by one or more marker values 1006 generated after assaying and analyzing the sample 1004. A computing device 1008 implementing the diagnostic framework 1018 will analyze and diagnose the subject 1002 as healthy, having sarcoidosis, or in some embodiments, having tuberculosis. The diagnosis is published to a user via a graphical user interface 1026.

In embodiments, to enhance security, subject privacy, and compliance with government regulations, subject data like the subject's marker values 1006 may be deleted after it is used to generate a computer assisted diagnosis. Thus, the sample information will no longer exist as standalone information on the one or more computing devices 1028 implementing the diagnostic framework 1018. Thus, the only subject data available to the computing device 1008 will be integrated into the diagnosis provided by the one or more computing devices.

In an illustrative diagram 1028 of the computing device 1008, the computing device 1008 may contain one or more processing unit(s) 1012 and memory 1014, both of which may be distributed across one or more physical or logical locations. The processing unit(s) 1012 may include any combination of central processing units (CPUs), graphical processing units (GPUs), single core processors, multi-core processors, application-specific integrated circuits (ASICs), programmable circuits such as Field Programmable Gate Arrays (FPGA), and the like. One or more of the processing unit(s) 1012 may be implemented in software and/or firmware in addition to hardware implementations. Software or firmware implementations of the processing unit(s) 1012 may include computer- or machine-executable instructions written in any suitable programming language to perform the various functions described. Software implementations of the processing unit(s) 1012 may be stored in whole or part in the memory 1014.

Additionally, the functionality of the computing devices 1008 can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

Computing device 1008 may be connected to a network through one or more network connectors 1016 for receiving and sending information. The network may be implemented as any type of communications network such as a local area network, a wide area network, a mesh network, and ad hoc network, a peer-to-peer network, the Internet, a cable network, a telephone network, and the like. In embodiments, the computing device 1008 have a direct connection to one or more other devices (e.g. devices that output subject 1002 information, like marker values 1006, in electrical or electronic form) without the presence of an intervening network. The direct connection may be implemented as a wired connection or a wireless connection. A wired connection may include one or more wires or cables physically connecting the computing device 1008 to another device. For example, the wired connection may be created by a headphone cable, a telephone cable, a SCSI cable, a USB cable, an Ethernet cable, or the like. The wireless connection may be created by radio frequency (e.g., any version of Bluetooth, ANT, Wi-Fi IEEE 802.11, etc.), infrared light, or the like.

The computing device 1008 may be a supercomputer, a network server, a desktop computer, a notebook computer, a collection of server computers such as a server farm, a cloud computing system that uses processing power, memory, and other hardware resources distributed across multiple geographic locations, or the like. The computing device 1008 may include one or more input/output components(s) such as a keyboard, a pointing device, a touchscreen, a microphone, a camera, a display, a speaker, a printer, and the like.

Memory 1014 of the computing device 1008 may include removable storage, non-removable storage, local storage, and/or remote storage to provide storage of computer-readable instructions, data structures, program modules, and other data. The memory 1014 may be implemented as computer-readable media. Computer-readable media includes non-volatile computer-readable storage media, removable and non-removable media implemented in any method or technology for storage of information such as computer-readable instructions, data structures, program modules, or other data. Computer-readable storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device.

The computing device 1008 includes multiple modules that may be implemented as instructions stored in the memory 1014 for execution by processing unit(s) 1012 and/or implemented, in whole or in part, by one or more hardware logic components or firmware. The diagnostic framework 1018 is contained within the computing device 1008 and may be implemented as instructions stored in the memory 1014 for execution by the processing unit(s) 1012, by hardware logic components, or both.

A scoring module 1012 obtains from an external source an indication of the expression of the tested markers in a sample 1004 as one or more marker value(s) 1006. The marker values 1006 can be obtained from a microarray or any machine connected to the computing device 1008 either directly or through the network connectors 1016. The marker values 1006 may also be previously saved or stored on a separate computing device or computer-readable media prior to being transferred to the scoring module 1020. The marker values 1008 may also be inputted directly by a user, including a physician or laboratory technician, through any appropriate I/O method. Exemplary I/O methods include any methods making use of the previously mentioned input/output components such as a keyboard, camera, microphone, touchscreen, or scanner.

The scoring module 1020 also obtains a reference level corresponding to the one or more marker values 1006. As with the marker values 1006, the reference levels can be calculated, as previously explained, and stored in a reference level database 1024, on the computing device 1008. Those having skill in the art will appreciate, however, that the one or more reference levels 1024 may, in other embodiments, be obtained either directly or through the network connectors 1016 from one or more separate computing devices, machines, or computer readable media. The reference levels may also be directly inputted by the user.

The scoring module 1020 may partially process, normalize, rewrite, anonymize, or otherwise modify the marker values 1006 or reference levels 1024. The scoring module 1020 will generate a score based at least in part on the one or more marker values 1006. In some embodiments this score is equivalent to the one or more marker values. In other embodiments, the score will be generated based at least in the part on the marker values 1006 and a weight associated with each corresponding marker. For example, markers with higher sensitivity, specificity, or both could be weighted more heavily than markers with lower sensitivity or specificity. Alternative scores may be generated based on any other previously discussed analytic process.

The scoring module 1020 provides the generated score to a diagnostic module 1022. The diagnostic module compares the score to the reference level and diagnoses the subject 1002 based on a result of the comparison as having sarcoidosis, not having sarcoidosis, or in some embodiments, having tuberculosis. The diagnosis is published to the user via a graphical user interface 1026.

Illustrative Process: For ease of understanding, the processes discussed in this disclosure are delineated as separate operations represented as independent blocks. However, these separately delineated operations should not be construed as necessarily order dependent in their performance. The order in which the process is described is not intended to be construed as a limitation, and any number of the described process blocks may be combined in any order to implement the process, or an alternate process. Moreover, it is also possible that one or more of the provided operations is modified or omitted.

An illustrative process is illustrated in 1100 for diagnosing sarcoidosis. At 1102, one or more reference levels are received, as well as an indication of the expression of relevant markers in a sample. The indication of the one or more marker values may be received from a clinician who assayed the sample for the value, or they may be received from a database where the values from a previously performed assay have been stored. At 1104, a score is generated at least partly based on the marker value. The score may be the same as the marker value, or it may be additionally based on a weight corresponding to each tested marker, or based in part on any other previously disclosed analytic process. Note that there may be a score for each marker, or there may be a single score based on an aggregation of data related to multiple marker values. At 1106, the score is compared to one or more reference levels. At 1108, a subject is diagnosed based on a result of the comparison 1106 as being healthy, having sarcoidosis, or in some embodiments, having tuberculosis.

In embodiments, the subjects diagnosed with sarcoidosis or tuberculosis using the methods disclosed herein can be effectively treated with the appropriate therapy. As an example, treating subjects with sarcoidosis includes delivering therapeutically effective amounts of an appropriate drug to alleviate one or more symptoms of sarcoidosis or tuberculosis.

### Particular Exemplary Embodiments Include:

1. A method of diagnosing sarcoidosis in a subject including assaying a sample derived from a subject for the presence of one or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and diagnosing the subject as healthy or having sarcoidosis based on the up- or down-regulation of the one or more markers, as compared to a reference level for each marker.

2. The method of embodiment 1 including assaying the sample for the presence of CFL1, 4FLI_A, ITPR3, CCL22, DSP, RAB36, PAR4, RGC32, DPY19L2, ERBB4, NEXMIF, or 1ZZP; and diagnosing the subject as healthy or having sarcoidosis based on the up- or down-regulation of the one or more markers.

3. The method of embodiment 1 including assaying the sample for the presence of IL7A, SH3YL1, RAB12, TRG10, POLKB, or INADL; and diagnosing the subject as healthy or having sarcoidosis based on the up- or down-regulation of the one or more markers.

4. The method of embodiment 1 including assaying the sample for the presence of two, three, four, five, six, seven, eight, nine, ten, eleven, twelve, thirteen, or more of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and diagnosing the subject as healthy or having sarcoidosis based on the up- or down-regulation of the one or more markers.

5. The method of any one of embodiment 1-4, further including assaying the sample for the presence of at least one of CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; and diagnosing the subject as healthy or having sarcoidosis based on the up- or down-regulation of the one or more markers.

6. A kit for diagnosing sarcoidosis in a subject wherein the kit includes a protein that binds one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; and a detectable label.

7. The kit according to embodiment 6 including one or more proteins that bind one of CFL1, 4FLI_A, ITPR3, CCL22, DSP, RAB36, PAR4, RGC32, DPY19L2, ERBB4, NEXMIF, or 1ZZP; and a detectable label.

8. The kit according to embodiment 6 including one or more proteins that bind IL17A, SH3YL1, RAB12, TRG10, POLKB, or INADL; and a detectable label.

9. The kit according to embodiment 6 including two, three, four, five, six, seven, eight, nine, ten, eleven, twelve, thirteen, or more proteins that each one of bind of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL, and a detectable label.

10. The kit according to embodiment 6, further including one or more proteins that bind CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv01890; BfrA; DAB2; or TCEB2; and a detectable label.

11. The kit according to any one of embodiments 6-10 wherein the proteins include antibodies, epitopes or mimotopes.

12. A kit for diagnosing sarcoidosis in a subject wherein the kit includes a nucleic acid that binds a gene encoding CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; and a detectable label.

13. The kit according to embodiment 12 including one or more nucleic acids that bind a gene encoding CFL1, 4FLI_A, ITPR3, CCL22, DSP, RAB36, PAR4, RGC32, DPY19L2, ERBB4, NEXMIF, or 1ZZP; and a detectable label.

14. The kit according to embodiment 12 including one or more nucleic acids that bind a gene encoding IL7A, SH3YL1, RAB12, TRG10, POLKB, or INADL; and a detectable label.

15. The kit according to embodiment 12 including two, three, four, five, six, seven, eight, nine, ten, eleven, twelve, thirteen, or more nucleic acids each of which binds a gene encoding one of CFL1, 4FLI_A, ITPR3, CCL22, DSP, RAB36, PAR4, RGC32, DPY19L2, ERBB4, NEXMIF, 1ZZP, IL_17A, SH3YL1, RAB12, TRG10, POLKB, and INADL; and a detectable label.

16. The kit according to embodiment 12, further including one or more nucleic acids that bind a gene encoding CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; and a detectable label.

17. The kit according to any one of embodiments 6-16 wherein the detectable label is a radioactive isotope, enzyme, dye, fluorescent dye, magnetic bead, or biotin.

18. The kit according any one of embodiments 6-17 wherein the kit further includes reagents to perform an enzyme-linked immunosorbent assay (ELISA), a radioimmunoassay (RIA), a Western blot, an immunoprecipitation, an immunohistochemical staining, flow cytometry, fluorescence-activated cell sorting (FACS), an enzyme substrate color method, and/or an antigen-antibody agglutination.

19. A method of diagnosing sarcoidosis in a subject including: obtaining a sample from a subject; assaying the sample for one or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; obtaining a value based on the assay; comparing the value to a reference level; and diagnosing the subject as healthy or having sarcoidosis based on the up- or down-regulation of the one or more markers as demonstrated by the value and the reference level.

20. The method according to embodiment 19 including assaying the sample for one or more markers selected from CFL1, 4FLI_A, ITPR3, CCL22, DSP, RAB36, PAR4, RGC32, DPY19L2, ERBB4, NEXMIF, and 1ZZP.

21. The method according to embodiment 19 including assaying the sample for one or more markers selected from IL17A, SH3YL1, RAB12, TRG10, POLKB, and INADL.

22. The method according to any one of embodiments 19-21 including assaying the sample for two, three, four, five, six, seven, eight, nine, ten, eleven, twelve, thirteen, or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL.

23. The method according to any one of embodiments 19-22, further including assaying the sample for one or more markers selected from CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2.

24. The method according to any one of embodiments 1-5 or 19-23, wherein assaying the sample for one or more markers include contacting the sample with a probe including a detectable label, wherein the probe binds the marker.

25. The method of any one of embodiments 1-5 or 19-24, wherein obtaining a value based on the assay includes analyzing the binding of the probe to the marker in the sample.

26. The method of any one of embodiments 1-5 or 19-25, wherein analyzing the binding of the probe to the marker in the sample includes quantitating the amount of the marker in the sample.

27. The method of any one of embodiments 1-5 or 19-26, wherein the sample is a tissue sample, a cell sample, a whole blood sample, a serum sample, a plasma sample, a saliva sample, a sputum sample, or a urine sample.

28. The method of any one of embodiments 1-5 or 19-27 wherein the value is a score.

29. The method of any one of embodiments 1-5 or 19-28 wherein the score is a weighted score.

30. A microarray including one or more proteins each of which binds one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL.

31. A microarray including one or more proteins each of which binds one of CFL1, 4FLI_A, ITPR3, CCL22, DSP, RAB36, PAR4, RGC32, DPY19L2, ERBB4, NEXMIF, or 1ZZP.

32. A microarray including one or more proteins each of which binds one of IL17A, SH3YL1, RAB12, TRG10, POLKB, or INADL.

33. The microarray of any one of embodiments 30-32, further including one or more proteins each of which binds one of CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv01890; BfrA; DAB2; or TCEB2.

34. A microarray including a nucleic acid that binds to a gene encoding CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL.

35. A microarray including a nucleic acid that binds a gene encoding CFL1, 4FLI_A, ITPR3, CCL22, DSP, RAB36, PAR4, RGC32, DPY19L2, ERBB4, NEXMIF, or 1ZZP.

36. A microarray including a nucleic acid that binds a gene encoding: IL17A, SH3YL1, RAB12, TRG10, POLKB, or INADL.

37. The microarray of any one of embodiments 34-36, further including at least one nucleic acid that binds a gene encoding CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2.

38. A microarray including one or more of the following proteins or a identifying peptide therefrom CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL.

39. A microarray including one or more of the following proteins or a identifying peptide therefrom CFL1, 4FLI_A, ITPR3, CCL22, DSP, RAB36, PAR4, RGC32, DPY19L2, ERBB4, NEXMIF, or 1ZZP.

40. A microarray including one or more of the following proteins or a identifying peptide therefrom: IL17A, SH3YL1, RAB12, TRG10, POLKB, or INADL.

41. The microarray of any one of embodiments 38-40, further including one or more of the following proteins or a identifying peptide therefrom: CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv01890; BfrA; DAB2; or TCEB2.

42. The microarray of any one of embodiments 30-41, wherein the protein or the nucleic acid on the microarray includes a label that can be detected.

43. The microarray of any one of embodiments 30-33 or 38-41, wherein the microarray includes two or more, three or more, four or more, five or more, six or more, seven or more, eight or more, or nine or more of the proteins on the microarray.

44. The microarray of any one of embodiments 34-37, wherein the microarray includes two or more, three or more, four or more, five or more, six or more, seven or more, eight or more, or nine or more of the nucleic acids on the microarray.

45. A kit comprising the microarray of any one of claims 30-44.

46. A kit according any one of embodiments 6-18 or 45, wherein the kit utilizes at least one clone or marker sequence identified herein, and wherein the kit comprises reagents to perform an enzyme-linked immunosorbent assay (ELISA), to detect specific immunoglobulin (IgG, IgA and Ig M).

47. A method of serological diagnosis of sarcoidosis, and/or a method of distinguishing sarcoidosis from other granulomatous diseases (such as tuberculosis), comprising detecting one or more immunoglobulin (IgG, IgA and Ig M) specific for and/or immunoreactive to at least one clone or marker sequence identified herein.

### Example 1. Systems and Methods to Diagnose Sarcoidosis and Identify Markers of the Condition

Significance. Aberrant immune responses are a major cause of a vast array of human diseases. Sarcoidosis is an inflammatory disease of unknown etiology sharing similarities with non-infectious and infectious granulomatous diseases, including Mycobacteria tuberculosis. Tuberculosis (TB) remains a major global health problem. There is a tremendous need to develop accurate tests to diagnose sarcoidosis and TB. A highly sensitive and specific T7 phage antigen library derived from bronchoalveolar lavage cells and leukocytes of sarcoidosis subjects was developed. This complex cDNA library was biopanned and a microarray was constructed to immunoscreen sera from healthy, sarcoidosis and TB subjects. A panel of specific antigens to classify sarcoidosis from healthy controls and subjects with TB was identified.

The research described in this Example is presented in U.S. Pat. No. 10,781,489 as well as applications related thereto; each of those applications and patent(s) are incorporated herein by references as though present herein. In particular, the Figures referenced in this Example can be found in U.S. Pat. No. 10,781,489.

Introduction. Sarcoidosis is an inflammatory granulomatous disease of unknown etiology affecting multiple organs, such as lungs, skin, CNS, and eyes. Common features shared by patients with sarcoidosis are the presence of non-caseating granuloma, a lack of cutaneous reaction to tuberculin skin testing (PPD) and increased local and circulating inflammatory cytokines. In addition, there is evidence of abnormal immune function that presents as cutaneous anergy accompanied by hypergammaglobulinemia. Sarcoidosis shares striking clinical and pathological similarities with infectious granulomatous diseases, especially Mycobacteria tuberculosis (MTB). Iannuzzi et al., N. Engl. J. Med. 2007; 357(21): 2153-65; Prince et al., J. Allergy Clin. Immunol. 2003; 111(2 Suppl): S613-23. Although there is mounting evidence of the presence of nonviable bacterial components (including MTB and Propionibacterium acnes) in sarcoidosis tissue (Gupta et al., Eur. Respir. J. 2007; 30(3): 508-16; Chen et al., Am. J. Respir. Crit. Care Med.; 181(4): 360-73; Negi et al., Modern pathology: an official journal of the United States and Canadian Academy of Pathology, Inc. 2012; 25(9): 1284-97) all attempts to isolate viable MTB or other microbial pathogens from sarcoidosis tissue have failed. Hunninghake et al., Sarcoidosis Vasc Diffuse Lung Dis 1999; 16(2): 149-73; Chen et al., J. Immunol. 2008; 181(12): 8784-96.

Intradermal injection of the Kveim-Siltzbach suspension (a granulomatous splenic tissue suspension) induces granuloma formation weeks later in sarcoidosis patients suggesting the presence of antigen(s) in granuloma tissue and host immunoreactivity to these antigens. Proteomics, genomics, transcriptomics, and high throughput technology clearly suggest that early immune reaction to diverse antigens is highly prevalent in a large number of rheumatic, neoplastic, and inflammatory diseases such as sarcoidosis. Several studies using state-of-the-art technologies have attempted to identify sarcoidosis antigens or to identify the underlying genetic and environmental factors (Hajizadeh et al., J. Clin. Immunol. 2007; 27(4): 445-54; Chen et al., Proc. Am. Thorac. Soc. 2007; 4(1): 101-7; Zhang et al., Respiratory research 2013; 14: 18) yet unifying environmental or genetic factors as initiators of this disease have not been found. Hunninghake et al., Sarcoidosis Vasc Diffuse Lung Dis 1999; 16(2): 149-73; Dubaniewicz, Autoimmunity reviews 2010; 9(6): 419-24; Eishi et al., J Clin Microbiol 2002; 40(1): 198-204; Oswald-Richter & Drake, Semin Respir Crit Care Med 31: 375-379, 2010. These studies reported a number of markers or variations in gene expression signatures, which, however, failed to discriminate between sarcoidosis and other inflammatory or granulomatous diseases. Koth et al., Am. J. Resp. Crit. Care 2011; 184(10): 1153-63; Maertzdorf et al., Proc. Natl. Acad. Sci. USA 2012; 109(20): 7853-8. This is partly due to the fact that several inflammatory diseases may respond to various antigens with activation of a similar transcriptome and/or inflammatory gene expression profiles.

Because non-caseating granulomas, cutaneous anergy and hypergammaglobulinemia suggest an immune dysfunction in this disease, it was hypothesized that sarcoidosis is triggered by a group of unknown antigens represented in the host immune cells. To identify the elusive antigen(s), a heterologous cDNA library derived from bronchoalveolar cell (BAL) samples and total white blood cells (WBC) from sarcoidosis patients was developed. Both sarcoid-derived libraries were then combined with cultured human monocytes and embryonic lung fibroblast cDNA libraries to build a complex sarcoidosis library (CSL). Furthermore, antibody recognition and random plaque selection was used during biopanning of the cDNA libraries to minimize the confounding effects of autoantibodies unrelated to sarcoidosis. It was tested whether this novel library representing relevant antigens could specifically recognize high IgG titer in sera of sarcoidosis subjects. This approach has been successfully applied in biomarker discovery for the diagnosis of lung, head and neck and breast cancer. Fernandez-Madrid et al., Cancer research 2004; 64(15): 5089-96; Fernandez-Madrid et al., Clinical cancer research: an official journal of the American Association for Cancer Research 1999; 5(6): 1393-400; Lin et al., Cancer epidemiology, biomarkers & prevention: a publication of the American Association for Cancer Research, cosponsored by the American Society of Preventive Oncology 2007; 16(11): 2396-405. A feature that distinguishes the described methods from previous studies is that the exquisite power of antibody recognition present in the sera of sarcoidosis patients was used to interrogate the potential antigens presented in the macrophages and monocytes.

The present study describes a novel approach to identify sarcoidosis antigens and to detect serum antibodies on high-throughput arrays. Sera from 3 cohorts (sarcoidosis, controls, and TB) were used for immunoscreening. Using bioinformatics tools, a large number of biomarkers with high sensitivity and specificity that can discriminate among the sera of patients with sarcoidosis, healthy controls and MTB was identified. Using the integrative-analysis method that combines results from two independent trials, clones that significantly differentiate sarcoidosis from controls were identified. Similarly, clones that differentially react with TB sera and not with sarcoidosis or control sera were identified. Furthermore, the top 10 discriminating antigens for TB and sarcoidosis were sequenced and homologies were identified in a public data base. These data indicate that a unique library enabling the detection of highly significant antigens to discriminate between patients with sarcoidosis and tuberculosis was developed.

Materials and Methods. Chemicals. All chemicals were purchased from Sigma-Aldrich (St. Louis, MO) unless specified otherwise. LeukoLOCK filters and RNAlater were purchased from Life Technologies (Grand Island, NY). The RNeasy Midi kit was obtained from Qiagen, (Valencia, CA). The T7 mouse monoclonal antibody was purchased from Novagen (San Diego, CA). Alexa Fluor 647 goat anti-human IgG and AlexFluor goat anti-mouse IgG antibodies were purchased from Life Technologies (Grand Island, NY).

Patient selection. This study was approved by the Institutional Review Board at Wayne State University and the Detroit Medical Center. Patients were recruited at the center for Sarcoidosis and Interstitial Lung Diseases (SILD), which is a referral center for patients with sarcoidosis and other ILDs. Three sources of patient derived materials have been used in this study: A) a BAL cDNA library was derived from BAL cells obtained during diagnostic bronchoscopy from newly diagnosed patients with sarcoidosis (n=20); B) a leukocyte cDNA library were developed from sarcoidosis patients who were followed in outpatient setting with various stages of sarcoidosis (n=36); and C) sera collected from 3 groups: 1) healthy controls, who were volunteers recruited from the community; 2) subjects with biopsy confirmed sarcoidosis who were followed in an outpatient setting; and 3) sera from subjects with culture positive TB collected at the Detroit Department of Health and Wellness Promotion. Subjects were included who had a diagnosis of sarcoidosis as proven by tissue biopsy per guidelines (Costabel & Hunninghake, Eur Respir J 14(4):735-737, 1999) and have a negative PPD. TB subjects were included who had a positive TB culture and were HIV negative. Subjects were excluded, who were positive for HIV or were receiving high dose immune suppressive medication that was defined as prednisone more than 15 mg alone or in combination with immune modulatory medications. Subjects who had positive PPD or quantiferon test were excluded from the sarcoidosis group. All study subjects signed a written informed consent.

Bronchoalveolar lavage: BAL cells were obtained, after informed consent, during diagnostic bronchoscopy from subjects with active sarcoidosis as previously described. Rastogi et al., American journal of respiratory and critical care medicine 2011; 183(4): 500-10. BAL cells were suspended in 500 μl of RNAlater and stored at −80° C.

Collection of total leukocytes from sarcoid subjects. Leukocytes from 36 sarcoid subjects were isolated using whole blood with LeukoLOCK filters as previously described. Glatt et al., Current pharmacogenomics and personalized medicine 2009; 7(3): 164-88.

Human macrophage (EL-1) and human lung embryonic fibroblast (MRC-5) cell cultures. Both cell lines were obtained from ATCC and cultured as per ATTC recommendations. From each cell line 1-2 mg RNA was isolated to construct the cDNA library.

Serum collection. Using standardized phlebotomy procedures blood samples were collected and allowed to clot and then centrifuged at 2500 rpm for 10 min. Supernatants were stored at −80° C.

Construction of T7 phage display cDNA libraries. Total RNA was isolated using the RNeasy Midi kit (Qiagen, Valencia, CA). Integrity of the RNA samples was assessed using the Agilent 2100 bioanalyzer. Total RNA, in the amount of 1-2 mg, was subjected to two cycles of polyA purification to minimize ribosomal RNA contamination as suggested by the manufacturer (Qiagen, Valencia, CA). The construction of phage cDNA libraries was performed using Novagen's Orient Express cDNA Synthesis (Random Primer System) and Cloning system as per manufacturer's suggestions (EMD Biosciences-Novagen). Each library was cloned using modified linkers that allow identification of the phage clones. Chatterjee et al., Cancer research 2006; 66(2): 1181-90. The number of clones in each of the 4 libraries was titrated by plaque assay as per manufacturer's instructions (EMD Biosciences-Novagen). Finally, the same number of phages from each BAL, WBC, EL-1 and MRCS library was pooled to generate a complex sarcoid library (CSL).

Biopanning of T7 phage displayed cDNA library with human sera. Differential biopanning for negative and positive selection was performed using sera from healthy controls to remove the non-specific IgG, and sarcoidosis sera for selective enrichment according to manufacturer's suggestions (T7 Select System, TB178; EMD Biosciences-Novagen). Protein G Plus-agarose beads (Santa Cruz Biotechnology) were used for serum IgG immobilization. Four rounds of biopannings were performed and the selected phage libraries were used for microarray immunoscreening. Each cycle of biopanning included passing the entire phage library through protein G beads coated with IgG from pooled sera of healthy controls, then passing through beads coated with IgGs from individual serum of sarcoid subjects. Microarray construction and immunoscreening. Informative phage clones were randomly picked and amplified after several rounds of biopannings and their lysates were arrayed in quintuplicates onto nitrocellulose FAST slides (Grace Biolabs, OR) using the ProSys 5510TL robot (Cartesian Technologies, CA). The nitrocellulose slides were then blocked with a solution of 1% BSA in PBS for 1 hour at room temperature followed by another hour of incubation with serum at a dilution of 1:300 in 1×PBS or plasma at a dilution of 1:100 as primary antibodies, together with mouse anti-T7 capsid antibody (0.15 μg/mL) and BL21 E. coli cell lysates (5 μg/mL). BL21 E. coli cell lysates were added to remove antibodies specific to E. coli from the serum. The microarrays were then washed three times at room temperature with a solution of PBS/0.1% Tween20 for 4 minutes. Secondary antibodies included goat anti-human IgG Alexa Fluor 647 (red fluorescent dye) 1 μg/mL and goat anti-mouse IgG Alexa Fluor 532 (green fluorescent dye) 0.05 μg/mL. After 1 hour incubation in the dark, the microarrays were washed 3 times with a solution of PBS/0.1% Tween20 for 4 minutes at room temperature, and 2 times in PBS for 4 minutes at room temperature and then air dried.

Sequencing of phage cDNA clones. Individual phage clones were PCR amplified using T7 phage forward primer and reverse primer and sequenced by Genwiz (South Plainfield, NJ), using T7 phage sequence primer.

Data acquisition and pre-processing. Following the immunoreaction, the microarrays were scanned in an Axon Laboratories 4100 scanner (Palo Alto, CA) using 532 and 647 nm lasers to produce a red (Alexa Fluor 647) and green (Alexa Fluor 532) composite image. Using the ImaGene 6.0 (Biodiscovery) image analysis software, the binding of each sarcoid specific peptide with IgGs in each serum was then analyzed and expressed as a ratio of red-to-green fluorescent intensities. The microarray data were further read into the R environment v2.3.0 (Team RDC. R: a language and environment for statistical computing. R Foundation for Statistical Computing; Vienna (Austria). 2004) and processed by a sequence of pre-processing, including background correction, omission of poor quality spots and log 2 transformations. Within array loess normalization was performed for each spot and summarized by median of triplicates and followed by between array quantile normalization.

Statistical analysis. A microarray analysis was performed using sera from sarcoid and healthy controls in two independent sets of experiments. Technical and biological sources of variation were expected in the design of the experiment. As opposed to pooling all datasets, one powerful and robust method is to integrate results from individual datasets. Obtaining a higher confidence list of markers than by using individual datasets was expected. To detect differentially expressed antigens between sarcoidosis samples and healthy controls, an integrative analysis of two datasets was performed. Limma's empirical Bayes moderated t-test identified fold-changes in expression of antigens that differed significantly between sarcoidosis and controls for each dataset separately. Then an integrative-analysis method—an adaptively-weighted method with one-sided correction (AW-OC) (Li & Tseng, The Annals of Applied Statistics 2011; 5(2A): 994-1019) was performed to combine the statistics from both datasets. The integrative method was designed to test whether an antigen is consistently up- or down regulated in sarcoidosis subjects in both datasets. False Discovery Rate (FDR) was estimated using the Benjamini-Hochberg method (Benjamini & Hochberg. J. R. Stat. Soc. Ser. B 57: 289-300, 1995).

To identify a panel of markers that classify sarcoidosis samples and controls, a strategy of univariate marker selection followed by multivariate modeling was used. The top antigens differentially expressed in the two groups were selected using the above described AW-OC approach. The top genes that were consistently up- or down-regulated in both datasets were used. The top markers were then required by the supervised classification models to achieve the most sensitivity and specificity in differentiating sarcoid and controls. The multivariate classification models chosen for this study were K-nearest neighbors (KNN) and support vector machine (SVM). The cross-validation technique was used to prevent the overfitting of data analysis due to a large number of antigens used to discriminate between sarcoid and control subjects. The study was performed in two nested 10-fold cross-validation loops, an inner loop to select the optimal number of antigens and an outer loop to measure the optimized model performance with estimation of the area under the receiver operating characteristic (AUROC) sensitivity and specificity. The receiver operating characteristic curves were estimated through 10-fold cross-validation. A moderated t-test was carried out to identify the significant clones between healthy controls, sarcoidosis and tuberculosis.

Results. Generation of cDNA libraries representative of sarcoidosis antigens. Both PBMCs and alveolar macrophages (AMs) play an important role in initiation of sarcoidosis granuloma. It has been shown that extracts from sarcoidosis BAL cells and peripheral blood monocytes (PBMCs) are able to initiate a Kveim-like reaction. Siltzbach & Ehrlich, The American Journal of Medicine 1954; 16(6): 790-803; Holter et al., The American Review of Respiratory Disease 1992; 145(4 Pt 1): 864-71. Therefore, total BAL cells and WBCs from patients with biopsy proven sarcoidosis were used to develop a cDNA antigen library. BAL cells and WBC were used as sources of antigens in order to increase the diversity of sarcoidosis antigens. To increase the chance of identifying sarcoidosis antigen(s), RNA was isolated from BAL samples obtained from 20 patients with active sarcoidosis to generate the BAL cDNA library. The patients' characteristics are shown in Table 1 (left panel). The LeukoLock system was used to isolate RNA from total leukocytes (WBC) obtained from a different cohort of 36 sarcoidosis subjects to build the WBC cDNA library. The patients' characteristics are shown in Table 1 (right panel).

TABLE 1
Subject Demographics, Chest X-Ray
Stages, and organ involvements

| BAL derived RNA | | | Leukocyte derived RNA | | |
|---|---|---|---|---|---|
| Age (Mean ± SEM) | 30 ± 8 | | Age (Mean ± SEM) | 36 ± 11.2 | |
| BMI (Mean ± SEM) | 27.7 ± 8.7 | | BMI (Mean ± SEM) | 31 ± 5.4 | |
| Gender, N (%) | | | Gender, N (%) | | |
| Male | 7 | (33) | Male | 12 | (33) |
| Female | 13 | (67) | Female | 24 | (67) |
| Race, N (%) | | | Race, N (%) | | |
| African American | 17 | (87) | African American | 32 | (88) |
| White | 3 | (13) | White | 4 | (12) |
| CXR stage, N (%) | | | CXR Stage, N (%) | | |
| 1 | 2 | (6) | 1 | 1 | (3) |
| 2 | 14 | (67) | 2 | 13 | (41) |
| 3 | 4 | (27) | 3 | 12 | (37) |

| | | | | |
|---|---|---|---|---|
| 4 | 0 | 4 | 6 | (19) |

| | | | |
|---|---|---|---|
| Lung | 18 | Lung | 33 |
| Extrapulmonary | 16 | Extrapulmonary | 31 |
| Neuro-ophthalmologic | 6 | Neuro-ophthalmologic | 11 |
| Skin | 6 | Skin | 13 |
| Liver | 2 | Liver | 4 |
| Heart | 1 | Heart | 2 |
| Prednisone | 1 | Prednisone | 3 |
| IMD | 0 | IMD | 14 |
| Smoking | | Smoking | |
| None | 12 | None | 26 |

Age, BMI and disease duration values are presented as means and variability in SD or range where indicated. N = Number of patients and percent shown in parentheses. IMD = Immunomodulatory drugs

Two other sources of cDNA, one from cultured human splenic monocytes (EL-1) and another from lung embryonic fibroblasts (MRCS) were used to generate two additional libraries. These sources were added to increase the chance of discovering potential sarcoidosis antigens. Each cDNA underwent two cycles of PolyA selection to minimize ribosomal contamination. These four libraries were developed as described in the Materials and Methods section. Each library was cloned using modified linkers; ECOR1/HindIII was used for BAL cDNA, ALA for WBC cDNA, LEU for MARC5 cDNA and THR for ED cDNA (FIG. 6 of U.S. Pat. No. 10,781,489). The use of these linkers enabled identification of the original library for each antigen.

Differential biopanning of sarcoidosis phage cDNA display libraries. The four phage cDNA display libraries (BAL, WBC, EL-1 and MARC5) were combined to generate a complex sarcoidosis library (CSL). To isolate a large panel of antigens, differential biopanning of the T7 phage cDNA display library was performed on the combined complex sarcoid library. A negative biopanning selection was done using 10 pooled sera from healthy controls to remove non-specific IgG, while 2 sarcoidosis sera were used for positive selective enrichment. One serum was obtained from a woman (P51) with systemic sarcoidosis who had uveitis and another serum was collected from a male subject (P197) who had active systemic sarcoidosis with renal involvement. Both patients had pulmonary involvements. Each clone was derived either from P51 or from P197. The titer of the complex library was assessed (FIG. 7A of U.S. Pat. No. 10,781,489) and individual phage clones were amplified by PCR (FIG. 7B of U.S. Pat. No. 10,781,489).

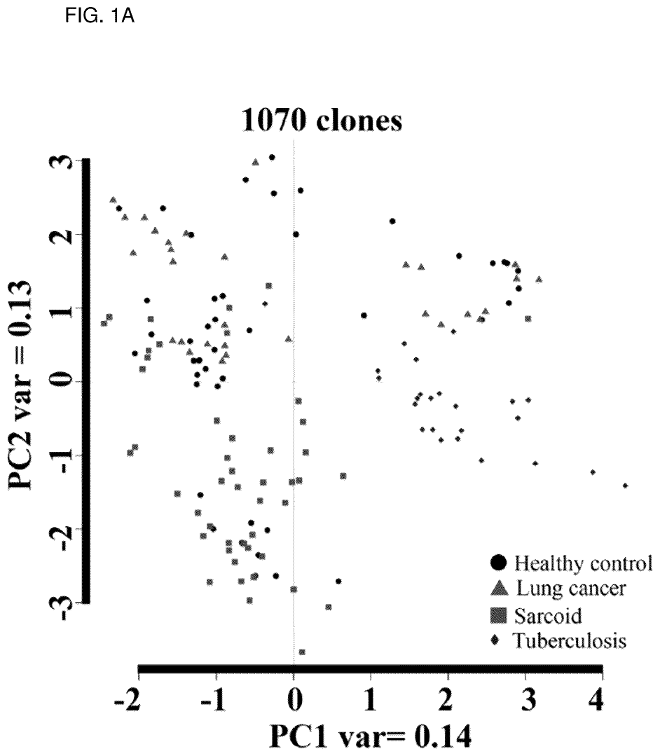

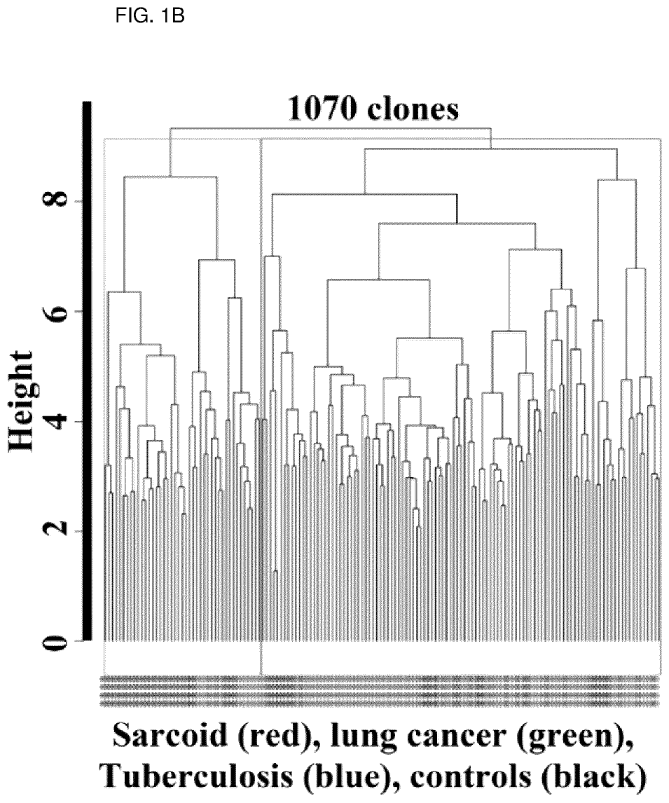

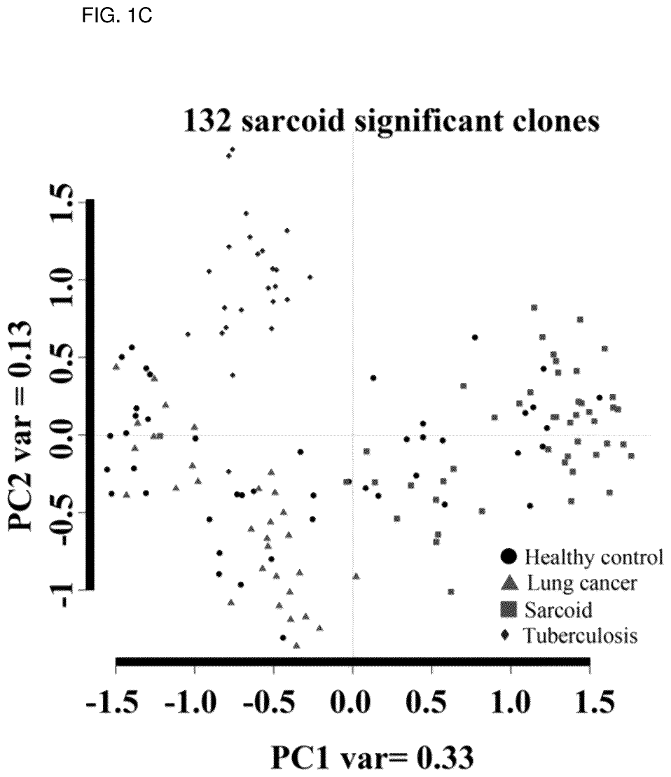

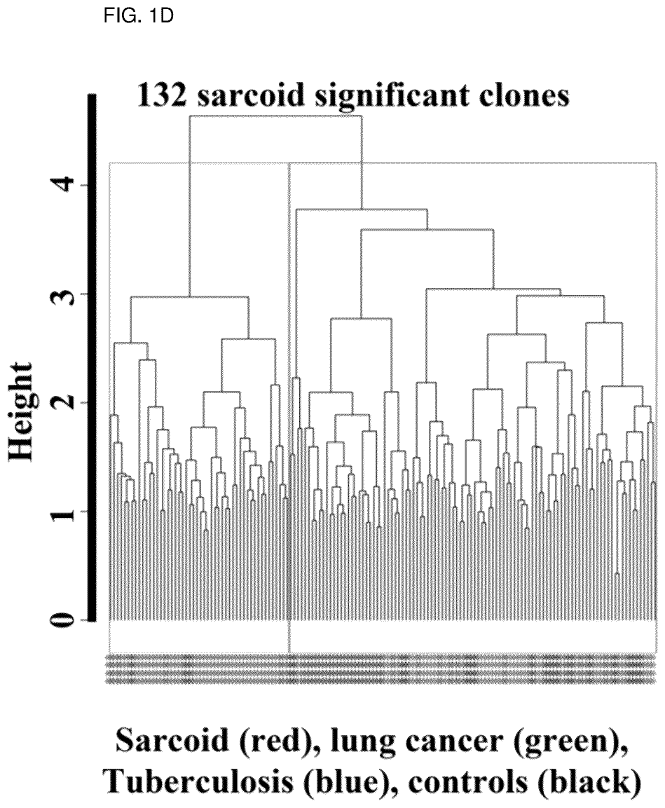

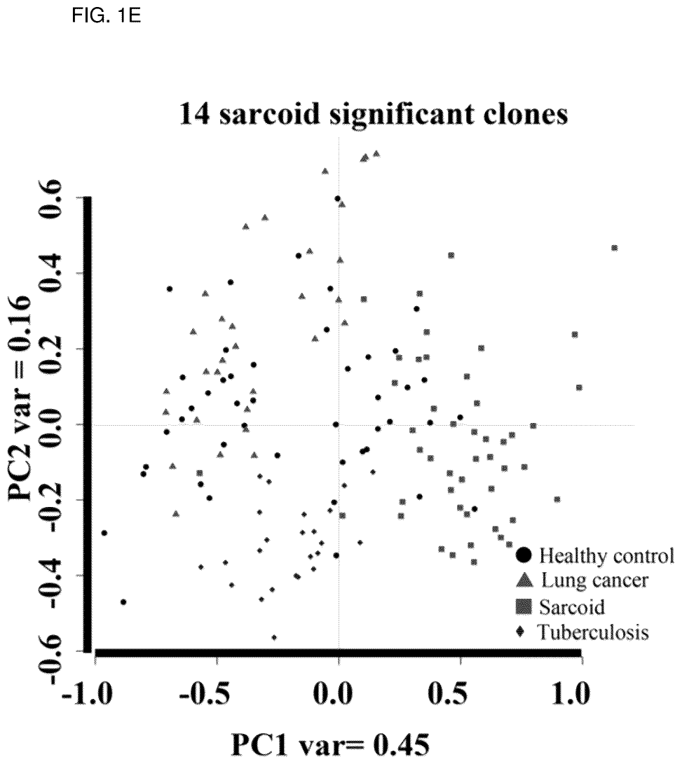

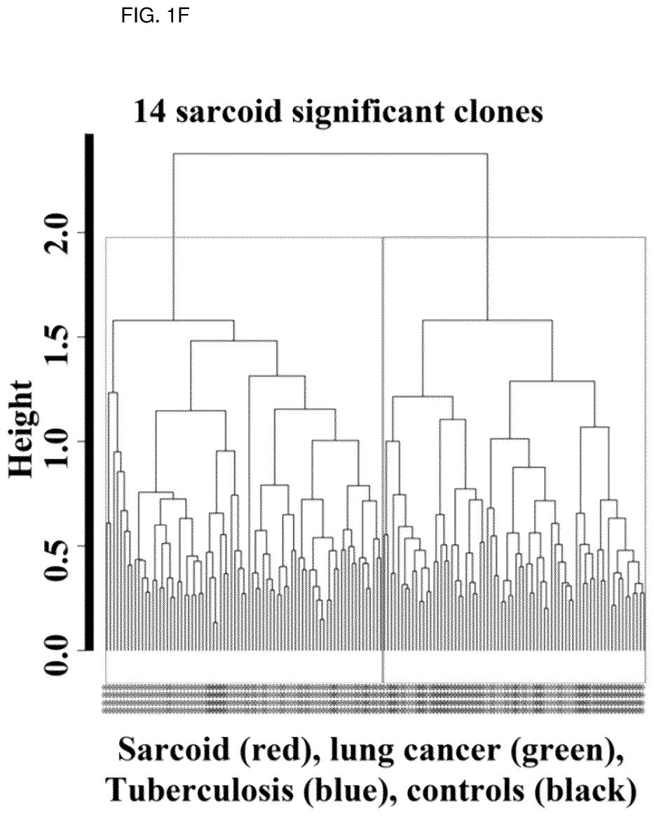

High-throughput protein microarray immunoreaction to select sarcoidosis specific antigens. A total of 1152 potential antigen antigens were randomly selected from the two highly enriched pools of T7 phage cDNA libraries (FIG. 1 of U.S. Pat. No. 10,781,489). These antigen antigens were robotically spotted on nitrocellulose Fast slides and were hybridized with sera of sarcoidosis patients or healthy controls. The binding of each of the arrayed potential sarcoidosis-specific peptides with antibodies in sera was quantified with Alexa Fluor 647 (red-fluorescent dye)-labeled goat anti-human antibody. The amount of phage particles at each spot throughout the microarray was detected using a mouse monoclonal antibody to the T7 capsid protein and quantified using Alexa Fluor 532 (green-fluorescent dye)-labeled goat anti-mouse antibody (FIG. 1 of U.S. Pat. No. 10,781,489). To correct for any small variation in the amount of antibody binding in each spot that may be due to different amounts of phage spotted on the microarray, the ratio of intensity of Alexa Fluor 647 over Alexa Fluor 532 was calculated for each spot. Following immunoreaction, the microarray data were processed by a sequence of transformations and then analyzed. The intra-assay reproducibility was assessed by comparing the results among five replicates printed within the same chip for each clone.

Selection of a panel of antigens and estimation of neural network classifier performance in sarcoidosis. A novel aspect of the described work was the integration of data from two independent trials of printing allowing the development of two data sets obtained from two independent cohorts of sarcoidosis patients and healthy controls utilized for hybridization. To generate the first dataset, sera from 54 sarcoidosis subjects and 45 healthy controls were immune-screened against 1152 sarcoidosis specific peptides. In a second dataset, sera from 19 healthy controls and 61 sarcoidosis subjects were similarly immune-screened with 1152 potential sarcoidosis specific antigens. Sera used in both data sets for hybridization had not been previously used for biopanning or selection of clones. Table 2 shows the clinical characteristics of sarcoidosis and healthy control subjects.

TABLE 2

| | Patient characteristics | | Control Subjects | |
|---|---|---|---|---|
| Age | 29.7 ± 13.4 y | | 33 ± 7.4 | |
| BMI | 29 ± 10.4 | | 28 ± 3.6 | |
| Gender, N | | | | |
| Female | 87 | (75) | 48 | (75) |
| Male | 28 | (25) | 16 | (25) |
| Race, N | | | | |
| African American | 107 | (89) | 44 | (69) |
| White | 8 | (11) | 20 | (31) |
| CXR stage, N | | | | |

| | | | |
|---|---|---|---|
| 0 | 3 | (2) | NA |
| 1 | 18 | (15) | NA |
| 2 | 49 | (43) | NA |
| 3 | 45 | (39) | NA |
| Organ Involvements, | | | |
| Neuro-ophthalmologic | 33 | (28) | NA |
| Lung | 109 | (94) | NA |
| Skin | 50 | (45) | NA |
| Multiorgan | 70 | (52) | NA |

Some Patients had multiple organ involvements
NA = Not Applicable

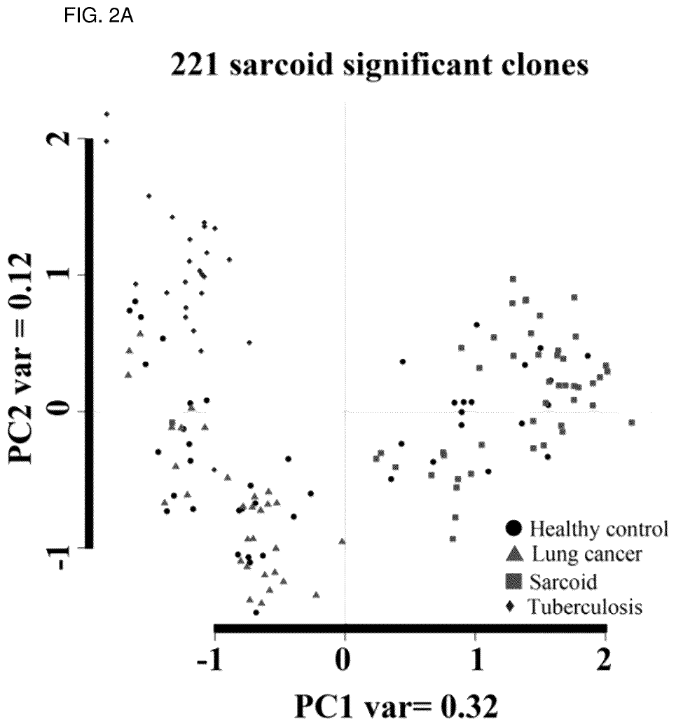

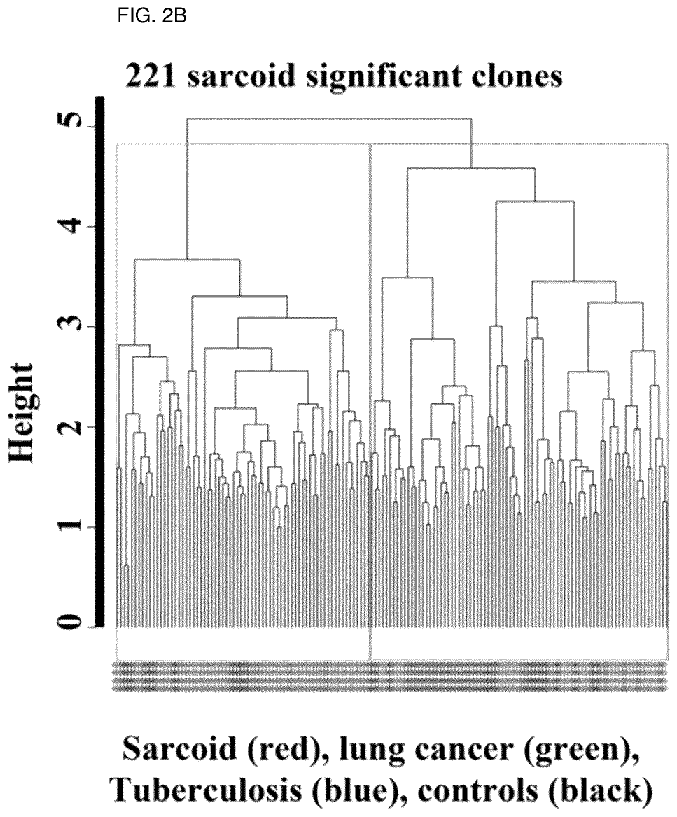

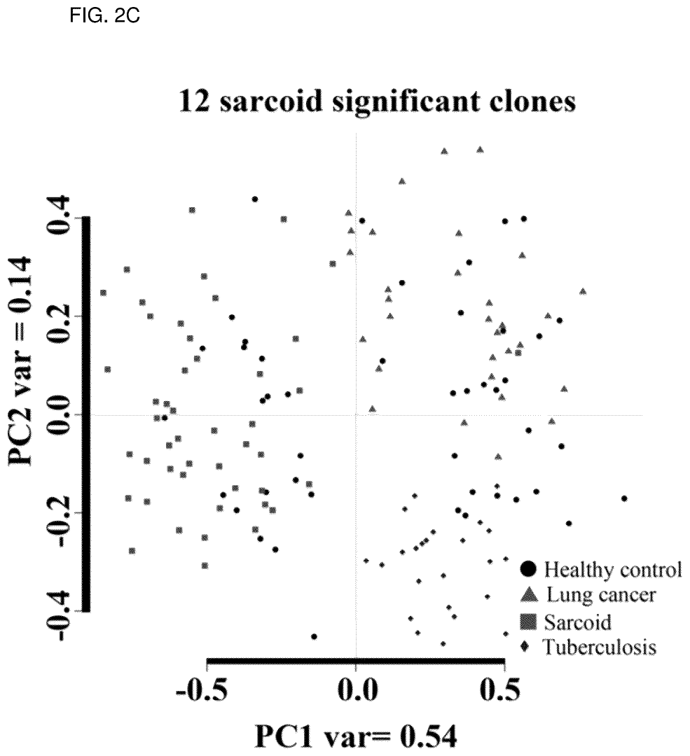

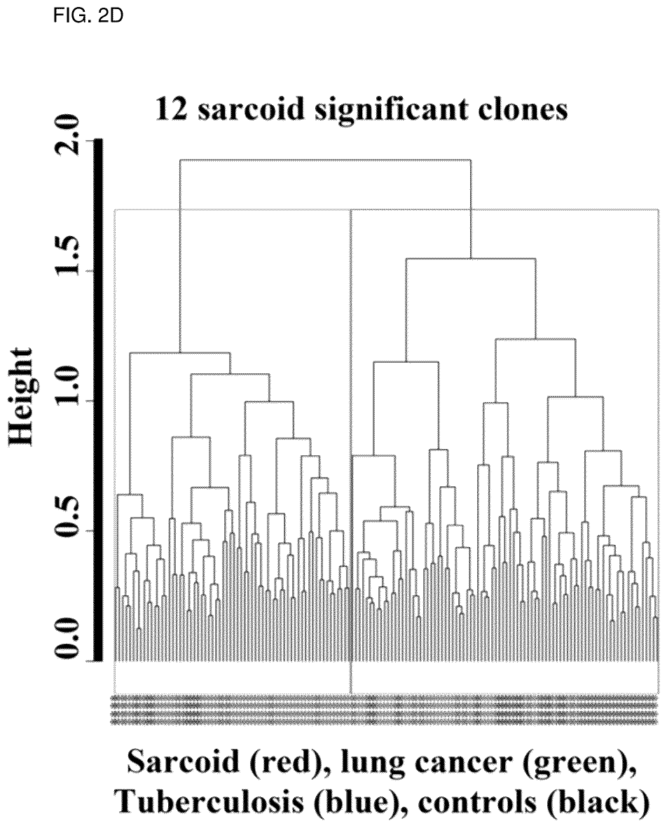

Within array loess normalization was performed for each spot and summarized by median of triplicates and followed by between array quantile normalization. After preprocessing, 1101 antigens common in both datasets were used for further analysis. Univariate and multivariate analyses were performed. Limma's empirical Bayes moderated t-test was used to identify fold-changes in expression of antigens that differed significantly between sarcoidosis and controls for each dataset separately. Then both datasets were combined using an integrative-analysis method—an adaptively-weighted method with one-sided correction (AW-OC). Li & Tseng, The Annals of Applied Statistics 2011; 5(2A): 994-1019. Out of the 1101 potential antigen, 259 showed a strong differentiation between sarcoidosis and healthy control subjects with adjusted p value (q value) <0.05 and FDR (false discovery rate)<0.05. FIG. 2A of U.S. Pat. No. 10,781,489 shows the heatmap of the 259 significant antigens that were differentially expressed in both datasets. Seventy eight markers out of 259 were consistently up- or down-regulated in sarcoidosis subjects. FIG. 2B of U.S. Pat. No. 10,781,489 shows the AUROC for this classifier. KNN method performed slightly better than SVM. Using the highly significant 32 antigens selected by AW.OC and KNN methods to classify sarcoidosis and healthy controls (AW.OC+KNN), the area under the curve (AUROC) was 0.78, with a sensitivity of 89% and a specificity of 83% estimated after 10-fold cross-validation (FIG. 2B of U.S. Pat. No. 10,781,489).

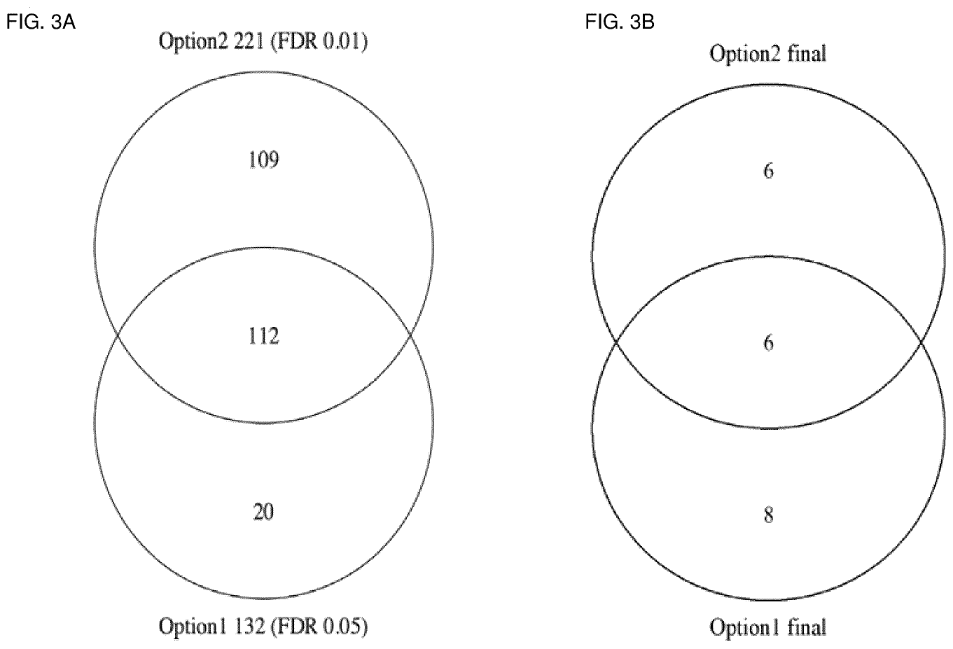

Characterization of 10 most significant sarcoid antigens. Based on the results of AW-OC integrative-analysis, the top 10 high performance antigens that predict sarcoidosis were identified. To further characterize the performance of each clone, the AU-ROC, and sensitivity and specificity given the optimal cutoff of the clones was calculated. FIG. 3 of U.S. Pat. No. 10,781,489 depicts the ROC curves for individual sarcoid antigens and their adjusted p value (q value). As shown, each antigen has a different specificity and sensitivity as well as ROC to predict the presence of sarcoidosis. ROC for these antigens ranged from the highest of 0.84 to the lowest of 0.7. Nine of 10 antigens were clearly up-regulated, whereas one was down-regulated. To further characterize the identified antigens, these 10 highest ranked antigens were sequenced. After obtaining the sequences of clones, the Expasy program was used to translate the cDNA sequences to protein sequences. Protein blast using Blastn and tblastn algorithms of the BLAST program were applied to identify the highest homology to identified proteins or peptides and these results were compared with corresponding nucleotide sequences using nucleotide blast. The predicted amino acid in frame with phage T7 gene 10 capsid proteins was also determined. Five Antigens (PC4, SAMDHI, DNAJC1, TPT1 and SH3YL1) among the top 10 fit the definition of an epitope containing known gene products in the reading frame known genes. The other five contained peptides coded by the inserted gene fragments leading to out of frame peptides, which fits the definition of mimotopes. Among the 10 high performance clones, nine were up-regulated and only one was down-regulated in sarcoidosis versus healthy controls. FIG. 8 of U.S. Pat. No. 10,781,489 shows the full length of proteins and genes of 10 sarcoidosis clones. Without being bound by theory, as sarcoidosis sera reacted to these out of frame peptides, it is likely that these clones represent sarcoidosis antigens produced as a result of altered reading frames or alternative splicing. Interestingly, when a similar technique was applied to discovery of cancer antigens, numerous out of frame peptides were discovered. Lin et al. (American Society of Preventive Oncology 16(11): 2396-405, 2007). Table 3 shows the 10 most significant sarcoidosis antigens, gene names and q-values.

TABLE 3

| | Up-Regulated in | | | Sensitivity// |
|---|---|---|---|---|
| | Sarcoidosis | | q Value | Specificity |
| Clone | Vs Healthy | Gene Name | AUC | %, 95% CI |
| P51_BP3_287 | Small inducible cytokine A21 | CCL21 | 1.9 × 10⁻²⁰ | 78//82 |
| (MRC5) | precursor | | 0.84 | |
| P51_BP3_281 | Methionine aminopeptidase 1 | Metap1 | 1.0 × 10⁻²⁰ | 70//82 |
| (BAL) | | | 0.78 | |
| P51_BP4_388 | Activated RNA polymerase II | PC4 | 0.00045 | 70//74 |
| (EL-1) | transcription cofactor variant 4 | | 0.75 | |
| P51_BP4_596 | RNA methyltransferase | CLI_3190 | 0.00045 | 72//74 |
| (WBC) | | | 0.72 | |
| P51_BP4_566 | Tumor necrosis factor | TNFR | 0.0009 | 70//71 |
| (WBC) | receptor superfamily member | SF21 | 0.74 | |
| | 21 precursor. Also known as | | | |
| | death receptor 6 (DR6) | | | |
| P51_BP3_283 | Monocyte differentiation | CD14 | 0.0009 | 68//65 |
| (WBC) | antigen CD14 | | 0.74 | |
| P51_BP3_47 | DnaJ (Hsp40) homolog | DNAJC1 | 0.002 | 60//82 |
| (EL-1) | subfamily C member 1 | | 0.72 | |
| | precursor | | | |
| P197_BP4_885 | Amyloid β A4 precursor | APBB1 | 0.007 | 75//82 |
| (BAL) | protein-binding family B | | 0.79 | |
| | member 1-interacting protein | | | |
| P51_BP4_577 | Fibroblast growth factor | FGFBP-2 | 0.009 | 64//68 |
| (BAL) | binding protein 2 precursor | | 0.70 | |
| | Up-Regulated in | | | Sensitivity// |
| | Sarcoidosis | | q Value | Specificity |
| Clone | Vs Healthy | Gene Name | AUC | %, 95% CI |
| P197_BP4_755 | SH3 domain-containing YSC84 | SH3YL1 | 1.0 × 10⁻²⁰ | 65//82 |
| (BAL) | like protein 1 | | 0.77 | |

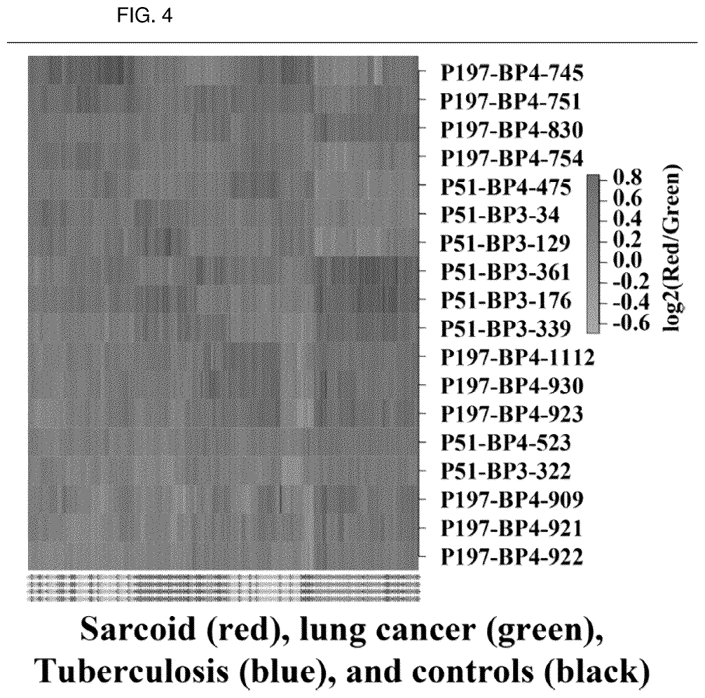

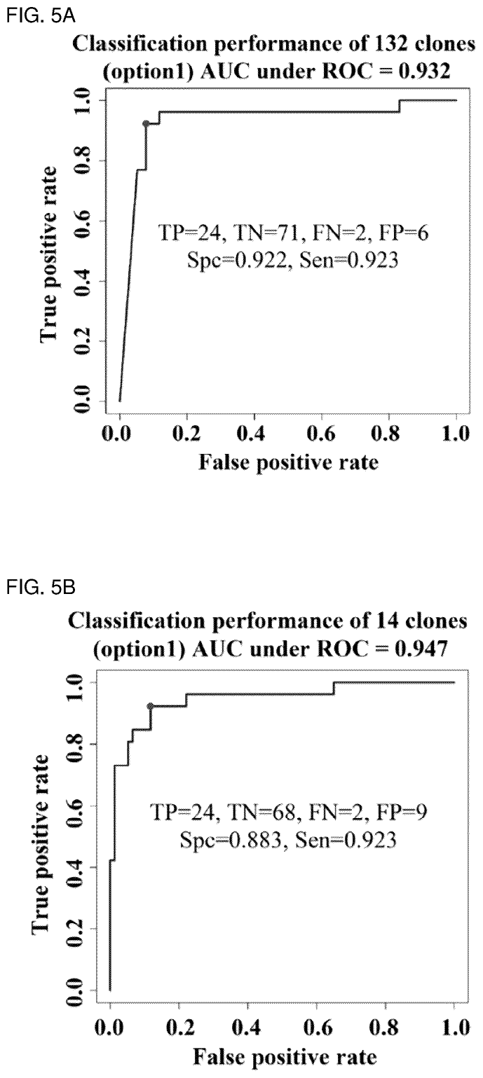

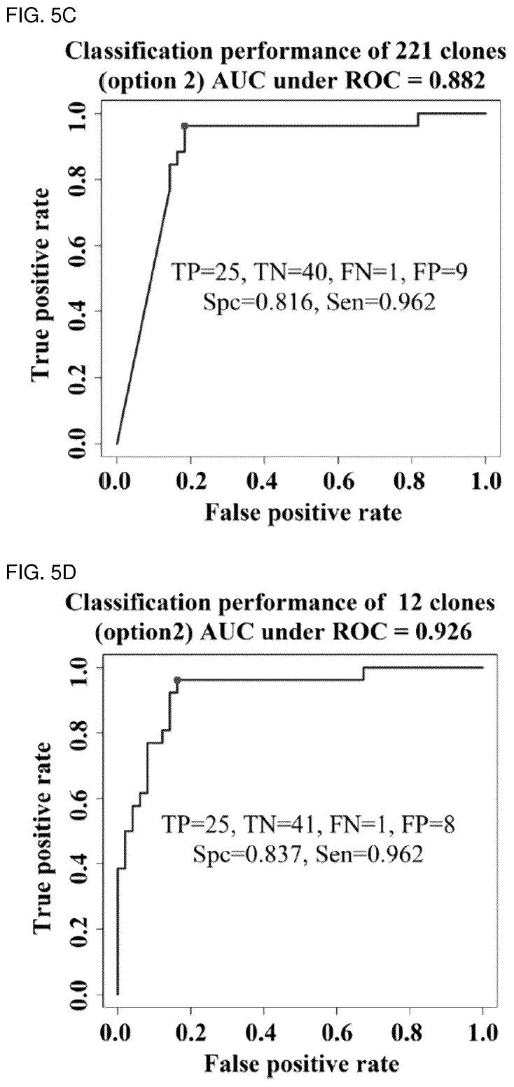

Complex sarcoidosis library detects novel antigens in the sera of tuberculosis patients. In view of the clinical and pathological similarities between MTB and sarcoidosis, a most useful clinical antigen(s) should discriminate between these two conditions. To this end, using the antigens identified by biopanning the CSL library a microarray was constructed, then this construct was interrogated with sera from 17 culture positive MTB subjects. Using a moderate t-test and a q value <0.05 in this system, 238 clones differentially expressed between TB and healthy controls and 380 clones differentially expressed between TB and sarcoidosis were identified. FIG. 4 of U.S. Pat. No. 10,781,489 shows a Venn diagram depicting the overlap between 259 sarcoidosis markers, 238 TB vs. control and 380 TB vs. sarcoidosis markers. Clearly, 47 clones differentiate both sarcoidosis and TB from healthy controls, while 5 of them cannot differentiate sarcoidosis from TB significantly. From these clones, 164 were found to be TB specific, and different from both healthy controls and sarcoidosis clones. FIG. 5 of U.S. Pat. No. 10,781,489 show the heatmap of 50 significant clones differentially expressed in all three groups. Similarly to the sarcoidosis antigens, the specificity and sensitivity of TB clones was analyzed to predict the presence of TB (Table 4). Finally, 10 TB antigens were sequenced and sequence homologies were searched using the same algorithm as previously described. Table 4 shows the 10 TB-specific antigens as compared to healthy controls as well as sarcoidosis.

TABLE 4

| | Up-Regulated in | | | | Sensitivity// |
|---|---|---|---|---|---|
| | TB vs Sarcoidosis | Gene | | | Specificity |
| Clone | Subjects | Name | q Value | AUC | %, 95% CI |
| P51_BP3_174 | Ferredoxin (Mycobacterium | Fed A | 4.9 × 10⁻¹⁵ | 0.87 | 88//83 |
| (MRC5) | tuberculosis) | | | | |
| P51_BP4_610 | WDFY3 protein (Homo | WDFY3 | 4.1 × 10⁻¹² | 0.92 | 88//84 |
| (BAL) | sapiens) | | | | |
| P51_BP3_266 | Membrane protein | MFS | 6.7 × 10⁻¹⁰ | 0.9 | 82//93 |
| (EL-1) | (Mycobacterium | | | | |
| | tuberculosis) | | | | |
| P51_BP3_166 | Leucine rich PPR-motif | LRPPRC | 1.3 × 10⁻⁹ | 0.81 | 71//90 |
| (BAL) | containing protein | | | | |
| | (Homo sapiens) | | | | |
| P51_BP4_704 | HLA-DR alpha (Homo | HLA-DR | 1.1 × 10⁻⁸ | 0.89 | 94//83 |
| (BAL) | sapiens) | | | | |
| P197_BP4_763 | Transketolase | TKT | 2.7 × 10⁻⁶ | 0.86 | 82//76 |
| (BAL) | (Mycobacterium | | | | |
| | tuberculosis) | | | | |
| P51-BP4_563 | Dihydroxy acid | Rv0189C | 1.04 × 10⁻⁶ | 0.85 | 76//86 |
| (BAL) | dehydratase | | | | |
| | (Mycobacterium | | | | |
| | tuberculosis) | | | | |
| | Down-Regulated in TB | | | | |
| Clone | vs Sarcoidosis Subjects | | | | |
| P51_BP3_113 | Chain A Mycobacterium | BfrA | 1.2 × 10⁻¹⁰ | 0.9 | 88//85 |
| (BAL) | tuberculosis | | | | |
| P51_BP3_200 | Disabled homolog 2 | DAB2 | 1.5 × 10⁻⁹ | 0.92 | 82//91 |
| (BAL) | isoform 2 (Homo | | | | |
| | sapiens) | | | | |
| P51_BP4_622 | Transcription | TCEB2 | 6.9 × 10⁻⁷ | 0.89 | 82//89 |
| (BAL) | elongation factor B | | | | |
| | polypeptide 2 isoform | | | | |
| | (Homo sapiens) | | | | |

After sequence analysis and homology search, one identical sequence between TB and sarcoidosis clone was identified. Although the identified clone's name was different: P51_BP3_287 versus P51_BP3_174, and they performed differently in sarcoidosis versus TB as indicated in q value (compare Table 3 and Table 4). However, using NCBI blast databases (mycobacterium toxoid and the universal blast) on the same sequence, two different proteins could be identified. FIG. 9 of U.S. Pat. No. 10,781,489 shows the full length of protein and genes of 10 TB antigens. Surprisingly, TB clones show much higher sensitivity and specificity; similarly the AUROC was larger for the majority of TB antigens (Table 4).

Discussion. The described work was inspired by the classic observation that the intradermal injection of a suspension of granulomatous splenic tissue (Kveim-Siltzbach test) induces granuloma formation weeks later in patients with sarcoidosis, suggesting the presence of antigen(s) in granuloma tissue and host immunoreactivity to those antigen(s). Kveim-like effects have also been observed using non-viable BAL cell extracts or PBMCs derived from sarcoidosis subjects. Several studies have attempted to identify specific antigens that can discriminate sarcoidosis from normal subjects or from patients with other granulomatous diseases such as TB (Hajizadeh et al., J. Clin. Immunol. 27(4): 445-54, 2007; Chen & Moller, Proc. Am. Thorac. Soc. 4(1): 101-7, 2007) but, most of these studies used limited proteomics or genomics to search for tissue antigens (Hajizadeh et al., J. Clin. Immunol. 27(4): 445-54, 2007; Richter et al., Am. J. Resp. Crit. Care 159(6): 1981-4, 1999; Song et al., J Exper Med 2005; 201(5): 755-67). Here, using novel high throughput technology, the current gap was overcome by constructing phage-protein microarrays in which peptides derived from a unique sarcoidosis cDNA library were expressed as a sarcoidosis phage fusion protein. The phage-protein microarrays were screened to identify phage-peptide clones that bind antibodies in serum samples from patients with sarcoidosis but not in those from controls. Importantly, the same microarray constructs were immune-screened using sera of culture positive TB patients.

The average length of identified peptides for sarcoidosis antigens was between 9-130 amino acids (AA), while the average peptide length for TB antigens was 9-209 AA. Among 10 sarcoidosis specific phage peptides, 5 expression sequence tags with in frame epitopes were identified. Five other reactive antigens were relatively short out of frame peptides meeting the criteria to be considered as mimotopes (mimetic sequence of a true epitope). Similarly, among 10 sequenced TB specific phage peptides, 5 in frame epitopes with full length in frame proteins with homology to known human sequences were identified. Five other sequences were relatively short peptides with homology to various known MTB proteins (Table 4).

Interestingly, TB antigens had much higher specificity and sensitivity as compared to antigens selective to sarcoidosis as indicated by higher AUCs (Table 4). Although the significance of mimotopes is not clear, it has been shown that some out of frame peptides are immunogenic and can activate MHC class I molecules. Due to smaller peptide sequences of mimotopes, they may have homology with diverse proteins. Prior studies using similar techniques in various cancers had similarly identified out of frame peptides. Lin et al., Cancer epidemiology, biomarkers & prevention: a publication of the American Association for Cancer Research, cosponsored by the American Society of Preventive Oncology 2007; 16(11): 2396-405; Wang et al., N. Engl. J. Med. 353(12): 1224-35, 2005; Chatterjee et al., Cancer Research 66(2): 1181-90, 2006. Detection of mimotopes in the described methods may be due to out of frame peptide synthesis secondary to altered ribosomal function, or may correspond to open reading frames, or generation of displayed peptides due to competition for binding during phage selection during phage insertion.

Although the primary goal was to identify the immune signature in sarcoidosis, a panel of antigens differentially expressed in sarcoidosis and tuberculosis as compared to healthy subjects was also identified. Tables 3 and 4 summarize the 10 most significant clones identified in sarcoidosis and tuberculosis respectively.

In recent years several groups have attempted to identify specific signatures to distinguish between tuberculosis and sarcoidosis using transcriptomics or gene expression profilings. Koth et al., Am. J. Resp. Crit. Care 2011; 184(10): 1153-63; Maertzdorf et al., Proc. Natl. Acad. Sci. USA 2012; 109(20): 7853-8; Berry et al., Nature 2010; 466(7309): 973-7. Yet most of these methods led to the discovery of a series of markers or expression signatures that failed to discriminate between these two diseases. Koth et al., American journal of respiratory and critical care medicine 2011; 184(10): 1153-63; Stone et al., PLoS One 2013; 8(1): e54487. This is partly due to the fact that several inflammatory or infectious diseases such as CD, lupus, sarcoidosis and tuberculosis may respond to various antigens with activation of similar transcriptomes and/or inflammatory gene expression profiles. For instance, Maertzdorf et al. found more similarity in the activated pathways than differences between sarcoidosis and MTB. Proc. Natl. Acad. Sci. USA 2012; 109(20): 7853-8. Their results in sarcoidosis were similar to those results by Berry indicating the importance of the interferon pathway (IFN) signature in MTB. Maertzdorf et al., Proc. Natl. Acad. Sci. USA 2012; 109(20): 7853-8; Berry et al., Nature 2010; 466(7309): 973-7. In addition, considerable pathway overlap was identified between lupus, sarcoidosis and TB. Maertzdorf et al., Proc. Natl. Acad. Sci. USA 2012; 109(20): 7853-8. However, despite similar genetic or transcriptomic signatures, these diseases are clinically entirely different and require different therapy. Tuberculosis, a global infectious disease caused by the intracellular bacterium Mycobacterium tuberculosis remains a worldwide health problem (online at who.int). One barrier for eradication of tuberculosis besides the lack of effective vaccination is the lack of reliable antigen to evaluate the activity of the disease and its response to treatment. Nahid et al., Am. J. Resp. Crit. Care 2011. 184(8): 972-9. Standard methods to diagnose TB and to monitor response to treatment rely on sputum microscopy and culture. The current CDC/NIH roadmap emphasizes the need for development of new TB antigens as alternative methods. Nahid et al., Am. J. Resp. Crit. Care e 2011. 184(8): 972-9. In view of this background, perhaps surprisingly, the described microarray platform could discriminate tuberculosis from sarcoidosis and healthy controls. In addition to antigens for sarcoidosis, more than 300 clones specifically for tuberculosis were detected. Interestingly, a considerable number of these clones were TB specific and related to bacterial growth of Mycobacterium tuberculosis, and its metabolism (Table 4). Recently a tremendous effort has been put toward elucidating the antibody response to MTB antigens, which has implications for the development of new antigens to diagnose and monitor successful treatment, as well as to develop effective vaccination. Kunnath-Velayudhan et al., Proc. Natl. Acad. Sci. USA 107(33): 14703-8, 2010. Yet, a consistent immune response to MTB has not been found. Most other studies searching for antigens in TB have identified unspecific markers primarily involving host response such as C-reactive protein or serum amyloid A and others, but not MTB specific antigens. Agranoff et al., Lancet 2006; 368(9540): 1012-21; De Groote et al., PLoS One 2013; 8(4): e61002. MTB has the ability to survive within host macrophages, largely escaping immune surveillance and maintaining its ability for replication and person to person transmission. Meena & Rajni, The FEBS J 2010; 277(11): 2416-27.

The primary goal of the described project was to discover antigens related to sarcoidosis. Yet, in addition specific antigens for TB were detected. These results are surprising, as the question remains, how can the sarcoidosis library detect TB specific antigens? Lungs are environmentally highly exposed to numerous bacteria, and the described library is predominantly derived from BAL cells that contain all types of immune cells, including macrophages that might have integrated messages from MTB. Without being bound by theory, this could be the reason why the CSL was able to detect TB specific antigens. Still, the major question is why BAL cells of patients with sarcoidosis can harbor MTB messages, yet respond to PPD skin testing with anergy, as all donors with sarcoidosis were PPD negative.

Similar to gene-expression profiling and the pattern-recognition approaches utilizing serum proteomics, the described methods may have the limitations of background signals, and sample-selection bias. To minimize these problems, an integrative-analysis method, an adaptively-weighted statistical method on two sets of data acquired in two independent experiments was applied. The discriminatory power of antibody signatures was validated by analyzing data from two completely different cohorts of patients.

In summary, a novel T7 phage display library derived from macrophages from BAL, monocytes from blood leukocytes of patients with sarcoidosis that may display a significant segment of the universe of potential sarcoidosis and MTB antigens that can be specially recognized by high IgG antibodies in sarcoidosis and MTB sera was developed. The described results support the hypothesis that sarcoidosis sera can recognize antigens presented in sarcoidosis materials. Current study of the antibody response can advance how proteomics can be used to harness immunity to identify and treat diseases, because it investigates antibody—antigen interactions and also evaluates the effects on antibody responses of pathogen and host characteristics.

### Example 2. Autoantibodies Against Cytoskeletons and Lysosomal Trafficking in Sarcoidosis Discriminate Sarcoidosis from Healthy Controls, Tuberculosis and Lung Cancers

Abstract Sarcoidosis is a granulomatous disease of unknown etiology and unifying environmental or genetic factors as initiators of this disease have not been found. Sarcoidosis subjects share several features, such as the presence of non-caseating granuloma, a lack of cutaneous reaction to tuberculin skin testing, and increased circulating cytokines. Other immunological features include a shift towards T helper type1 response, lymphopenia or neutropenia, and in some cases increased production of autoantibodies. Hypergammaglobulinemia is a frequent finding in sarcoidosis, which may suggest active humoral immunity to unknown antigen(s). To identify the role of autoantibodies, four different T7 phage display cDNA libraries were constructed, two of which originate from sarcoid BAL cells and WBCs. Two other cDNA libraries are derived from cultured human embryonic fibroblasts and splenic monocytes. After biopanning, 1117 sarcoidosis-specific clones that were arrayed were selected and immunoscreened with 152 samples from sarcoidosis and a diversified population. To identify the sarcoidosis classifiers two statistical approaches were undertaken: First, significant biomarkers between sarcoidosis and healthy controls were identified, and second approach identified sarcoidosis markers comparing sarcoidosis and all other groups. At the threshold of an FDR<0.01, 14 clones in the first approach and 12 clones in the second approach discriminating sarcoidosis from other groups in each option were identified (see Table 7). Furthermore, the classifiers were used to build a naïve Bayes model on the training set. The naïve Bayes performance was validated on an independent test set. Two statistical approaches yielded in two different ROC curves (AUC): The first approach yielded an AUC of 0.947 using 14 significant clones with a sensitivity of 0.93 and specificity of 0.88, whereas the AUC of the second option was 0.92 and a sensitivity of 0.96 and specificity of 0.83. These results suggest robust classifier performance.

These results show that sarcoidosis is associated with a specific pattern of immunoreactivity that can discriminate it from other diseases.

At least some of the research described in this Example was published on Jan. 20, 2022, as Hanoudi et al., Mol. Biomed. 3:3, 2022 (doi.org/10.1186/s43556-021-00064-x).

### INTRODUCTION

Sarcoidosis is a granulomatous disease of unknown etiology (1), yet the unifying environmental or genetic factors as initiators of this disease have not been found (2-5). Sarcoidosis affects multiple organs, such as the mediastinal lymph nodes, lungs, skin, CNS and the eyes (Costabel & Hunninghake, Eur Respir J 14: 735-737, 1999; Hunninghake et al., Sarcoidosis Vasc Diffuse Lung Dis 16: 149-173, 1999; Costabel, Eur Respir J Suppl 32: 56s-68s, 2001; Iannuzzi et al., N Engl J Med 357: 2153-2165, 2007). Other immunological features include a shift towards T helper type1 response, lymphopenia or neutropenia, and in some cases increased production of autoantibodies (Amital et al., Internat Arch Allergy Immunology 99: 34-36, 1992; Terunuma et al., Int. J. Dermatol. 39: 551-553, 2000; Kataria & Holter, Clin Chest Med 18: 719-739, 1997; Cuilliere-Dartigues et al., Am J Hematol 85: 891, 2010).

Sarcoidosis often coincides with other autoimmune disorders such as lupus erythematosus, vitiligo (Terunuma et al., 39: 51-553 2000), autoimmune hepatitis, and CD (Terunuma et al., 39: 51-553 2000; Marzano et al., Clin Exp Dermatol 21:466-467 1996; Nakayama et al., Intern Med 46:1657-1661, 2007; Rajoriya et al., Postgrad Med J 85: 233-237, 2009). Several studies have suggested that the cellular and humoral responses associated with granuloma formation in this disease are the consequence of an exaggerated immune response to unknown antigens (Gerke & Hunninghake, Clin Chest Med 29:379-390, 2008; Muller-Quernheim et al., Clin Chest Med29: 391-414, 2008). Hypergammaglobulinemia, widely regarded as non-specific, is a frequent finding in sarcoidosis that may suggest active humoral immunity to unknown antigen(s) (Kataria & Holter, Clin Chest Med 18:719-739, 1997). Furthermore, subjects with sarcoidosis share several features, such as the presence of non-caseating granuloma, a lack of cutaneous reaction to tuberculin skin testing, and increased local and circulating inflammatory cytokines (Costabel & Hunninghake, Eur Respir J14:735-737, 1999; Costabel, Eur Respir J Suppl 32:56s-68s, 2001; Iannuzzi et al., N Engl J Med 357:2153-2165, 2007). Interestingly, lack of responsiveness to PPD can also occur in other inflammatory diseases such as Crohn's disease (CD), rheumatoid arthritis (RA), or infectious diseases such as leprosy (Oswald-Richter & Drake, Semin Respir Crit Care Med 31: 375-379, 2010, Bianco & Spiteri, Clin Experi Immunol 110:1-3, 1997, Mow et al., Clin Gastroenterol Hepatol 2: 309-313, 2004). Pulmonary sarcoidosis and active pulmonary Tuberculosis (MTB) share a number of clinical, radiological and histological similarities making differential diagnosis difficult.

The prevalence of sarcoidosis is higher in the northern hemisphere. Furthermore, it has been reported that the incidence of sarcoidosis is increasing in the developing world and China (Babu, J Ophthal Inflam Infect 3:53, 2013, Li et al., Sarcoidosis Vasc Diffuse Lung Dis 29:11-18, 2012). Therefore, the development of highly accurate diagnostic classifiers for the diagnosis of sarcoidosis has significance worldwide. To identify the sarcoidosis-associated antigens, four different T7 phage display cDNA libraries were constructed, two of which originated from sarcoid BAL cells and WBCs. Two other cDNA libraries were derived from cultured human embryonic fibroblasts and splenic monocytes. All 4 libraries were combined into a complex library. This novel complex library is custom made for the discovery of biomarkers of respiratory disorders, in particular for sarcoidosis (Talwar et al., Viruses, 10, 2018; Talwar et al., Scientific Reports, 7:17745, 2017; Talwar et al., EBioMedicine, 2:341-350, 2015; Talwar et al., Mycobacterial Dis. 6(2):214, 2016). Recently, it was shown that the microarray technology detects specific classifiers for various respiratory diseases (Talwar et al, Viruses, 10, 2018; Talwar et al., Scientific Reports, 7:17745, 2017; Talwar et al., EBioMedicine, 2:341-350, 2015; Talwar et al., Mycobacterial Dis. 6(2):214, 2016). In previous work, applying the same technology, specific biomarkers were identified for sarcoidosis and Tuberculosis as well as cystic fibrosis. Here, the hypothesis that was tested was that this technology is able to identify the specific classifiers for sarcoidosis in early stages within a large heterogeneous group of study subjects, including, heathy controls, Tuberculosis and lung cancer.

### 2. Materials and Methods

Chemicals. All chemicals were purchased from Sigma-Aldrich (St. Louis, MO) unless specified otherwise. LeukoLOCK filters and RNAlater were purchased from Life Technologies (Grand Island, NY). The RNeasy Midi kit was obtained from Qiagen, (Valencia, CA). The T7 mouse monoclonal antibody was purchased from Novagen (San Diego, CA). Alexa Fluor 647 goat anti-human IgG and Alex Fluor goat anti-mouse IgG antibodies were purchased from Life Technologies (Grand Island, NY).

Patient selection. This study was approved by the institutional review board at Wayne State University, and the Detroit Medical Center. Sera were collected from 3 groups: 1) healthy volunteers; 2) sarcoidosis subjects, 3) patients with lung cancers; and 4) smear positive pulmonary TB patients. All sarcoidosis subjects were ambulatory patients. All study subjects signed a written informed consent. All methods were performed in accordance with the human investigation guidelines and regulations by the IRB (protocol No=055208MP4E) at Wayne State University. Sera from patients with Tuberculosis were obtained from the Foundation for Innovative New Diagnostics (FIND, Geneva, Switzerland). All TB patients had smear positive sputum for Mycobacterium Tuberculosis.

Serum collection. Using standardized phlebotomy procedures blood samples were collected and stored at −80° C. (Talwar et al., EBioMedicine, 2:341-350, 2015).

Construction and Biopanning of T7 phage display cDNA libraries. T7 phage display libraries from BALs, WBCs, EL-1 and MRCS were made to generate a complex sarcoid library (CSL) (Talwar et al., EBioMedicine, 2:341-350, 2015). Differential biopanning for negative selection was performed using sera from healthy controls to remove the non-specific IgG, and sarcoidosis sera for positive enrichment (Talwar et al., EBioMedicine, 2:341-350, 2015).

Microarray construction and immunoscreening. Informative phage clones were randomly picked and amplified after four rounds of biopannings and their lysates were arrayed in quintuplicates onto nitrocellulose FAST slides (Grace Biolabs, OR) using the ProSys 5510TL robot (Cartesian Technologies, CA). The nitrocellulose slides were hybridized with sera and processed as described previously (Talwar et al., EBioMedicine, 2:341-350, 2015)

Sequencing of phage cDNA clones. Individual phage clones were PCR amplified using T7 phage forward primer 5′ GTTCTATCCGCAACGTTATGG 3′ (SEQ ID NO: 19) and reverse primer 5′ GGAGGAAAGTCGTTTTTTGGGG 3′ (SEQ ID NO: 20) and sequenced by Genwiz (South Plainfield, NJ), using T7 phage sequence primer TGCTAAGGACAACGTTATCGG (SEQ ID NO: 21).

Data acquisition and pre-processing. Following the immunoreaction, the microarrays were scanned in an Axon Laboratories 4100 scanner (Palo Alto, CA) using 532 and 647 nm lasers to produce a red (Alexa Fluor 647) and green (Alexa Fluor 532) composite image. Cy5 (red dye) labeled anti-human antibody was used to detect IgGs in human serum that were reactive to peptide clones, and a Cy3 (green dye) labeled antibody was used to detect the phage capsid protein (Talwar et al., EBioMedicine, 2:341-350, 2015).

Using the ImaGene 6.0 (Biodiscovery) image analysis software, the binding intensity of each peptide with IgGs in sera was expressed as log 2 (red/green) fluorescent intensities. These data were pre-processed using the limma package in the R language environment (Talwar et al., Scientific Reports, 7:17745, 2017; Ritchie et al., Nucleic Acids Res, 43:e47, 2015; R Core Team, R Foundation for Statistical Computing, 2015) and normexp method was applied to correct the background (Talwar et al., Scientific reports, 7:17745, 2017; Ritchie et al., Bioinformatics, 23:2700-2707, 2007). Within array normalization was performed using the LOESS method (Talwar et al., EBioMedicine, 2:341-350, 2015; Ritchie et al., Bioinformatics, 23:2700-2707, 2007; Yang et al., Nucleic Acids Res, 30:e15-e15, 2002). The scale method was applied to normalize between arrays (Ritchie et al., Bioinformatics, 23:2700-2707, 2007; Yang et al., Nucleic Acids Res, 30:e15-e15, 2002). The intensity ratio of a clone in active sarcoidosis divided by the same clone intensity ratio from healthy control samples was calculated to determine the fold change of a clone.

### Statistical Analyses:

To detect differentially expressed antigens for sarcoidosis, a two-tailed t-test correcting for multiple comparisons using the false discovery rate (FDR) algorithm with a threshold of either 0.05 or 0.01 FDR (Costabel et al., Eur Respir, J 14:735-737, 1999) was applied. All significant clones were sorted in an increasing order. Two statistical analyses using two-tailed t-tests were applied. In Option 1, a t-test between sarcoidosis training samples versus healthy controls training samples was applied. Out of the 52 sarcoidosis samples, 26 samples were randomly assigned to the training set and the other 26 samples to the testing set. The training and testing set for the 45 healthy controls were randomly assigned to 23 samples in training and 22 samples in test sets. In the testing set, 24 tuberculosis samples and 31 lung cancer samples were added.

In Option 2, the samples were randomly split from all groups in half. The first half of 23 control, 26 sarcoidoses, 16 lung cancer, and 12 tuberculosis samples were assigned to the training set. The second half of 22 control, 26 sarcoidoses, 16 lung cancer, and 12 tuberculosis samples were assigned to the testing set. A t-test between sarcoidosis training samples versus healthy controls, lung cancer, and tuberculosis training samples were applied to identify significant clones. For both options, the performance of significant clones “classifiers clones” were assessed, by applying principal component analysis (RCA), agglomerative hierarchal clustering (HC), heatmap, and naïve Bayes classifier. The naïve Bayes classifier model was bunt on the training samples to predict sarcoidosis samples from others (healthy controls and tuberculosis and king cancer) samples and tested the classification model on the testing set (samples not used in the training set).

### Results:

A panel of potential antigens was randomly selected from two highly enriched pools of 17 phage cDNA libraries through biopanning of the CSL library (Hunninghake et al., Sarcoidosis Vasc Diffuse Lung Dis, 16:149-173, 1999; Dubaniewicz. Autoimmun Rev 9; 419-424, 2010). The constructed microarray platform was immunoscreened with 152 sera from diverse study subjects that included: healthy controls (n=45); sarcoidosis (n=52), smear-positive TB patients (n=24), and DEng cancer patients (n=31). The demographics of the study subjects are shown in (Table 5). Following immunoreaction, the microarray data were pre-processed and then analyzed as previously described (Talwar et al., Viruses, 10, 2018; Talwar et al., Scientific Reports, 7:17745, 2017; Talwar et al., EBioMedicine, 2:341-350, 2015). To identify significant sarcoidosis clones, two different t-tests were applied: i) sarcoidosis training samples vs. healthy control training samples (option 1), and ii) sarcoidosis training samples vs, the rest (healthy controls, LC and TB samples (option 2). Two options resulted in two sets of differentially expressed clones. The first set (option 1) identifies 132 significantly different clones (0.05≤FDR) between sarcoidosis and healthy controls.

TABLE 5
Subject Demographics

| | | | TB | Lung |
|---|---|---|---|---|
| Characteristic | Controls | Sarcoidosis | Subjects | Cancers |
| Age (Mean ± SEM) | 40 ± 7.5 | 30.6 ± 11.8 | 40.5 ± 8.5 | 62.3 ± 11.9 |
| Gender, N (%) | | | | |
| Male | 12 (26) | 11 (21) | 14 (58) | 12 (38) |
| Female | 33 (74) | 41 (79) | 10 (41) | 18 (58) |
| Race, N (%) | | | | |
| African American | 31 (69) | 49 (89) | | |
| African | — | | 4 (25) | 0 (0) |
| Caucasian | — | 3 (11) | | 31 (100) |
| Asians | 14 (31) | | 20 (75) | 0 (0) |
| BMI (Mean + SEM) | 27 ± 3.8 | 28 ± 10.5 | 28 ± 6.9 | 28 ± 9 |
| Organ involvement | | | | |
| Neuro- | NA | 31 (29) | — | NA |
| ophthalmologic | | | | |
| Lung | NA | 48 (96) | 24 (100) | 31(100) |
| Skin | NA | 46 (43) | — | NA |
| Multiorgan | NA | 45 (61) | — | NA |
| PPD ^{a} | NA | Negative | — | NA |
| TB smear ^{b} | NA | Negative | Positive | NA |

NA = Not applicable;
^{a} PPD = Mantoux test (purified protein derivative);
^{b} TB Smear obtained

Unsupervised principal component analysis (RCA) was performed using all 1070 clones with data from 152 study subjects. As shown in FIG. 1A, several healthy controls and sarcoidosis patients were clustered with TB and lung cancer groups. Also performed was unsupervised hierarchical clustering with all 1070 clones on these 152 samples. The magenta duster with a mix of samples and lacks specific sub-dusters of sarcoidosis samples (FIG. 1B) was observed. FIGS. 1A and 1B show that using all 1070 clones lacks the ability to cluster the sarcoidosis samples well.

To determine whether the 132 significant clones (FDR<0.05) and 14 clones from option 1 improved the class separation of sarcoidosis patients from healthy controls, TB samples and lung cancer, two RCA plots were constructed. As shown in FIG. 1C, using 132 significant clones aided in an improved class separation of sarcoidosis subjects from all other groups with a variance of 33% along with the PC1 (FIG. 1C). Similarly, using hierarchical clustering showed better separation of sarcoidosis samples from all the others (FIG. 1D). Decreasing the FDR threshold to 0.01, 14 highly significant clones differentially reactive in sarcoidosis versus healthy controls were identified. When a RCA plot was constructed by using the 14 final clones from option 1, it resulted in a clear class separation of sarcoidosis samples from TB patients, healthy controls and LC patients. The result in FIG. 1E shows Forty-five percent of variance was explained along the PC 1 when the clustering algorithm was performed using 14 sarcoidosis clones on all subjects. A distinct hierarchical linkage separating sarcoidosis samples from other samples was observed (FIG. 1F).

The option 2 approach yielded in 221 significant clones (0.01<FDR) differentiating sarcoidosis from all other conditions. To demonstrate the performance of the clones identified with option 2 (sarcoidosis samples versus all other samples), similarly, RCA and hierarchical clustering were applied. As shown in FIG. 2A, using 221 clones aided in an improved class separation of sarcoidosis subjects from all other groups with variance of 32% along with the PC1. Similarly, when the clustering algorithm was performed using 221 significant clones (FDR<0.01), a distinct hierarchical linkage nearly perfectly separating the sarcoidosis patients from TB and well separation from LC and healthy controls was observed (FIG. 2B). Top 12 reactive clones in option 2 were chosen to construct RCA plot and hierarchical clustering. As shown in FIGS. 2C and 2D, using the top 12 clones aided in an improved class separation of sarcoidosis subjects from all other groups with a variance of 54% along the PC1 (FIG. 2C). A distinct hierarchical linkage is well separating the sarcoidosis samples from all other samples. The clustering analysis using the top 14 clones using option 1, and the top 12 using option 2 show a robust clustering of sarcoidosis samples from the rest (healthy controls, TB and LC).

FIGS. 3A and B, illustrates the Venn diagram of significant clones yielded through two different statistical approaches as well as their intersection.

FIG. 4 displays a heatmap plot of the distinct expression features of the final classifier clones from options 1 and 2. The heatmap shows the profile for the final clones with all samples.

### Identification of Classifiers to Predict Sarcoidosis

To determine the classification performance of the identified clones using option 1 and 2, the naïve Bayes classification method was applied using option 1 and option 2 significant clones. Also assessed was the classification performance of the top 14 clones from option 1 and the top 12 clones from option 2. The classification models were trained on the training set and tested to classify sarcoidosis samples from other (healthy control, TB, and LC) on the testing set. As shown in FIG. 5A, the AUC under the ROC using the significant 132 clones (option 1) was 0.932 with true positive (TP) of 24, true negative (TN) of 71, False negative (FN) of 2 and false positive (FP) of 6. Next, the classifier model was applied on the test set using the top 14 clones from option 1, The results of this analysis are in FIG. 5B, which shows an improved AUC of 0.947 when compared with the classification model of the 132 significant clones. FIG. 5C, shows the classification results of the 221 significant clones (option 2) with an AUC under the ROC of 0.882 with TP of 25, TN of 40, FN of 1 and FP of 9, Similar to option 1, the classification model on the test set using the top 12 clones from option 2 was applied. The results of this analysis are in FIG. 5D, which it shows an improved AUC of 0.926 when compared with the classification model of the 221 significant clones. Those results suggest a robust classifier performance when using the top 14 clones from option 1 and the top 12 clones from option 2. See also Table 7.

Characterization of Sarcoidosis classifiers. Based on the results of training and test sets, the sarcoidosis classifier clones were characterized through sequencing. The classifiers' clones were sequenced and the Expasy program was applied to translate the cDNA sequences to peptide/protein sequences. Protein blast using algorithms of the BLAST program was applied to identify the highest homology to identified peptides (Talwar et al., Scientific Reports, 7:17745, 2017; Talwar et al., EBioMedicine, 2:341-350, 2015). Furthermore, these results were compared with corresponding nucleotide sequences using nucleotide BLAST and determined the predicted amino acids in frame with T7 phage 10B gene capsid proteins. The identified clones were blasted with human genomes and then selected those specific peptide sequences that have the highest homology of amino acids sequence. After sequencing, it was identified that two different DNA inserts were repeated twice. The selected peptide sequences of the final classifiers clones with the highest homology is shown in Table 6 shows the sarcoidosis clones identified by both statistical approaches (option 1 and option 2), gene names, sensitivity, specificity, and FDR adjusted p-values.

TABLE 6
Clone Characteristics/Classifiers

| | | | SEQ | | FDR ª | | | |
|---|---|---|---|---|---|---|---|---|
| Clone | | Gene | ID | | Corrected | | | |
| ID | Protein names | Name | NO: | p value | p value | AUC ^{b} | Sensitivity | Specificity |
| | Increased in Sarcoidosis | | | | | | | |
| P197- | Cofilin 1 (non-muscle), | CFL1 | 1 | 2.1E−11 | 8.10E−09 | 0.90 | 0.89 | 0.82 |
| BP4- | isoform CRA_a | | | | | | | |
| 922^{1&2} | | | | | | | | |
| P197- | Chain A, Human | 4FLI\|A | 2 | 1.51E−10 | 2.70E−08 | 0.82 | 0.81 | 0.71 |
| BP4- | Metap1 | | | | | | | |
| 921^{1&2} | | | | | | | | |
| P197- | Inositol 1,4,5- | ITPR3 | 3 | 6.94E−09 | 4.96E−07 | 0.80 | 0.85 | 0.63 |
| BP4- | trisphosphate receptor | | | | | | | |
| 923^{1&2} | type 3 | | | | | | | |
| P197- | C-C motif chemokine | CCL22 | 4 | 2.06E−05 | 3.68E−03 | 0.71 | 0.85 | 0.62 |
| BP4- | 22 precursor | | | | | | | |
| 1112¹ | | | | | | | | |
| P197- | Chain A, Desmoplakin | DSP | 5 | 6.62E−05 | 5.45E−03 | 0.82 | 0.81 | 0.75 |
| BP4- | | | | | | | | |
| 909¹ | | | | | | | | |
| P197- | Ras-related protein | RAB36 | 6 | 8.30E−05 | 6.30E−03 | 0.76 | 0.89 | 0.64 |
| BP4- | Rab-36 isoform 1 | | | | | | | |
| 930¹ | | | | | | | | |
| P51- | Apoptosis related | PAR4 | 7 | 1.40E−04 | 8.83E−03 | 0.75 | 0.81 | 0.70 |
| BP3- | protein APR-4, partial | | | | | | | |
| 176¹ | | | | | | | | |
| P51- | Response gene to | RGC32 | 8 | 7.94E−11 | 1.70E−08 | 0.86 | 0.89 | 0.76 |
| BP4- | complement 32, | | | | | | | |
| 523² | isoform CRA_b | | | | | | | |
| P51- | Probable | DPY19L2 | 9 | 1.1E−09 | 1.57E−07 | 0.75 | 0.89 | 0.59 |
| BP3- | C_mannosyltransferase | | | | | | | |
| 322² | DPY19L2 isoform X17 | | | | | | | |
| P51- | Receptor tyrosine- | ERBB4 | 10 | 6.95E−09 | 4.96E−07 | 0.78 | 0.92 | 0.65 |
| BP3- | isoform X1 | | | | | | | |
| 339² | | | | | | | | |
| P197- | protein kinase erbB-4 | | | | | | | |
| BP4- | | | | | | | | |
| 753¹ | | | | | | | | |
| P51- | Neurite extension and | NEXMIF | 11 | 1.94E−07 | 5.16E−06 | 0.84 | 0.81 | 0.84 |
| BP3- | migration factor | | | | | | | |
| 361² | | | | | | | | |
| P197- | Solution structure of the | 1ZZP | 12 | 3.83E−07 | 8.54E−06 | 0.90 | 0.85 | 0.86 |
| BP4- | F-actin binding domain | | | | | | | |
| 830² | of Bcr-Abl/c-Abl | | | | | | | |

Decreased in Sarcoidosis

| | | | | | | | | |
|---|---|---|---|---|---|---|---|---|
| P51- | Interleukin 17A | IL17A | 13 | 2.3E−09 | 2.74E−07 | 0.87 | 0.81 | 0.80 |
| BP3- | | | | | | | | |
| 129^{1&2} | | | | | | | | |
| P197- | SH3 domain-containing | SH3YL1 | 14 | 13.99E−09 | 4.01 E−07 | 0.84 | 0.73 | 0.88 |
| BP4- | YSC84-like protein 1 | | | | | | | |
| 745^{1&2} | isoform 4 | | | | | | | |
| P197- | Ras-related protein | RAB12 | 15 | 1.17E−09 | 1.57E−07 | 0.73 | 0.54 | 0.90 |
| BP4- | Rab-12 | | | | | | | |
| 754^{1&2} | | | | | | | | |
| P197- | Transformation-related | TRG10 | 16 | 2.50E−05 | 3.82E−03 | 0.67 | 0.65 | 0.69 |
| BP4- | protein 10 | | | | | | | |
| 751¹ | | | | | | | | |
| P51- | Beta-polymerase | POLB | 17 | 8.83E−05 | 6.30E−03 | 0.80 | 0.75 | 0.91 |
| BP4- | | | | | | | | |
| 475¹ | | | | | | | | |
| P51- | | | | | | | | |
| BP3-57¹ | | | | | | | | |
| P51- | INADL protein | INADL | 18 | 1.2E−08 | 7.11 E−07 | 0.83 | 0.89 | 0.65 |
| BP3-34² | | | | | | | | |

Key:
Clone ID: subscription ^{1&2}refers to the clone identified through Option 1 or Option 2.
ª False discovery rate;
^{b} Area under the curve.

### DISCUSSION

Patients with sarcoidosis exhibit other immunological features including a shift towards T helper type 1 response (Rastogi et al., Am J Respir Crit Care Med, 183:500-510, 2011), lymphopenia or neutropenia, hypergammaglobulinemia, and in some cases increased production of autoantibodies (Amital et al., Int Arch Allergy Immunol, 99:34-36, 1992; Terunuma et al., Int J Dermatol, 39:551-553, 2000; Kataria et al., Clin Chest Med, 18:719-739, 1997; Cuilliere-Dartigues et al., Am J Hematol, 85:891, 2010). Sarcoidosis often coincides with other autoimmune disorders such as lupus erythematosus, vitiligo (Terunuma et al., Int J Dermatol, 39:551-553, 2000), and autoimmune hepatitis (Terunuma et al., Int J Dermatol, 39:551-553, 2000; Marzano et al., Clin Exp Dermatol, 21:466-467, 1996; Nakayama et al., Intern Med, 46:1657-1661, 2007; Rajoriya et al., Postgrad Med J, 85:233-237, 2009). Hypergammaglobulinemia, widely regarded as non-specific, is a frequent finding in sarcoidosis that may suggest active humoral immunity to unknown antigen(s) (Kataria et al., Clin Chest Med, 18:719-739, 1997). Several studies have suggested that the cellular and humoral responses associated with granuloma formation in this disease are the consequence of an exaggerated immune response to unknown antigens (Gerke et al., Clin Chest Med, 29:379-390, 2008; Muller-Quernheim et al., Clin Chest Med, 29:391-414, 2008).

Numerous studies found components (RNA, DNA) of pathogens including Propionibacterium acnes and Mycobacterium tuberculosis in sarcoidosis tissues (Gerke et al., Clin Chest Med, 29:379-390, 2008; Muller-Quernheim et al., Clin Chest Med, 29:391-414, 2008; Eishi, Biomed Res Int, doi:10.1155/2013/935289, 2013; Brownell et al., Am J Respir Cell Mol Biol, 45:899-905, 2011; Mortaz et al., Int J Mycobacterial, 3:225-229, 2014; Kataria et al., Methods, 9:268-294, 1996). Similarly, it has been shown that sarcoidosis blood monocytes react to TB antigens including, ESAT6 and KatG with increased interferon gamma production (Oswald-Richter et al., J Clin Immunol, 30:157-166, 2010). In contrast to the individuals infected with TB, who respond to PPD with positive skin tests, sarcoidosis subjects are non-reactive to PPD skin tests. Using serological expression cloning (SEREX) as a basis, the relevant methods of biomarker discovery were examined and an innovative immunoscreening approach was developed to optimize the identification of specific molecular markers (Talwar et al., EBioMedicine, 2:341-350, 2015; Fernandez Madrid et al., Autoimmun Rev, 4:230-235, 2005; Lin et al., Cancer Epidemiol Biomarkers Prev, 16(11):2396-2405, 2007). To achieve this goal, a heterologous sarcoidosis antigens derived from RNA of numerous sarcoidosis subjects displayed on T7 phage (Talwar et al., EBioMedicine, 2:341-350, 2015; Talwar et al., Mycobact Dis, 6(2):214, 2016). Furthermore, antibody recognition and random plaque selection during biopanning of the libraries were used to minimize the confounding effects of nonspecific antibodies. Recent evidence indicates that panels of biomarkers can achieve significantly higher diagnostic accuracy than individual biomarkers (Fernandez Madrid et al., Autoimmun Rev, 4:230-235, 2005; Kolly et al., FEMS Microbiol Lett, 358:30-35, 2014; Wang et al., N Engl J Med, 353:1224-1235, 2005; Chatterjee et al., Cancer Biomark, 11:59-73, 2012; Chatterjee et al., Cancer Res, 66:1181-1190, 2006; Chatterjee et al., Methods Mol Biol, 520:21-38, 2009).

Previously, it was shown that the complex antigen library detects autoantibodies as biomarkers in sera of sarcoidosis, cystic fibrosis and MTB patients with high sensitivity and specificity as compared to healthy subjects (Talwar et al., Viruses, 10, 2018; Talwar et al., Scientific Reports, 7:17745, 2017; Talwar et al., EBioMedicine, 2:341-350, 2015). The current data indicates that the technology detects sarcoidosis classifiers as compared to various other lung diseases. Important to note that current sarcoidosis group differs from the previous study group. Sera were collected during initial diagnosis of sarcoidosis and none of patients were treated with corticosteroids or other immunosuppressive medications. Additionally, sera from TB patients differed from a previous study (Talwar et al., EBioMedicine, 2:341-350, 2015), as previous TB group used samples from patients who were treated with antituberculosis medication. Furthermore, two different statistical approaches to the data were performed: Option 1, first detected the significant biomarkers between healthy controls vs. sarcoidosis; whereas option 2 chose the sarcoidosis clones by comparing sarcoidosis samples vs. all other groups. In both options independent training and testing sets were used. Interestingly, 6 antigen clones were identical between option 1 and 2. Option 1 yielded in 8 unique clones, whereas option 2 yielded in 6 specific clones. Two sequences were repeated twice in two different clone IDs (Table 6).

Among 18 classifier clones, one clone (Chain A, Human Metap1) was repeated in both approaches. Importantly, this sequence was also identified as sarcoidosis specific clone (Talwar et al., EBioMedicine, 2:341-350, 2015). Another repeated clone has homology to SH3YL1. Previously, only a little was known about the role of SH3YL1 in human diseases or its role in the immunity, recent emerging data indicates that SH3YL1 regulates nicotinamide adenine dinucleotide phosphate (NADPH) oxidase (Nox) isozymes, thereby it modulates reactive oxygen species (Yoo et al., Cell Rep, 33:108245, 2020). Other reports suggest that this protein regulates endosomal sorting complex required for transport (ESCRT) that is involved in endosome-lysosomal trafficking (Hasegawa et al., J Cell Sci, 132, 2019). Further experiments need to elucidate the role of SH3YL1 in sarcoidosis. Two clones related to endo-lysosomal trafficking were identified: one is ras-related protein RAB-12 and another is ras-related protein Rab-36. Both of these proteins belong to Rab GTPase family. Recent evidence indicates the involvement of GTPase family in the complex of membrane trafficking from endosome and lysosome, as well as their essential roles in signaling that control the cell proliferation and differentiation (Stenmark, Nat Rev Mol Cell Biol, 10: 513-525, 2009). Additionally, a relatively large peptide sequence (43AA) was identified with sequence homology to transformation related protein. This gene encodes a member of the bone morphogenetic protein (BMP) receptor family of transmembrane serine/threonine kinases. The ligands of this receptor are members of the TGF-beta superfamily. BMPs are involved in endochondral bone formation and embryogenesis. These proteins transduce their signals through the formation of heteromeric complexes of two different types of serine (threonine) kinase receptors: type I receptors of about 50-55 kD and type II receptors of about 70-80 kD (Katagiri et al., Cold Spring Harb Perspect Biol, 8:a021899, 2016). For instance, mutations in BMP2 have been associated with primary pulmonary hypertension (Teichert-Kuliszewska et al., Circ Res, 98:209-217, 2006). Another clone antigen was the colony stimulating factor 1 (isoform CRA_b). CSF-1 signals through its receptor (CSF-1R) promotes the differentiation of myeloid progenitors into heterogeneous populations of monocytes, macrophages, dendritic cells, and bone-resorbing osteoclasts (Cannarile et al., J Immunother Cancer, 5:1-13, 2017).

Previously, a prominent role of monocytes and macrophages in sarcoidosis (Talreja et al., Front Immunol 11:779, 2020; Talreja et al., Elife 8, 2019) was shown. A relatively small sequence had homology with erbB-4 gene. This gene is a member of the tyrosine protein kinase family and the epidermal growth factor receptor subfamily and is one of the four members in the EGFR subfamily of receptor tyrosine kinases. Three important antigen clones were related to cytoskeleton. An antigenic peptide with 23 AA, which has homology to Cofilin 1, was identified. Cofilin family promotes actin filament disassembly and has been shown to be involved in myofibroblast differentiation (Pho et al., Am J Physiol Heart Circ Physiol, 294:H1767-H1778, 2008). Interestingly, when NCIB's protein BLAST was used for all species, including all microorganisms this sequence had high homology with flagellin. Further investigation is needed to elucidate the role of this peptide sequence in sarcoidosis including fibrotic changes associated with this disease. Another related clone to cytoskeleton was the Chain A, Desmoplakin (DSP). DSP is a key junctional protein necessary for the morphogenesis and integrity of epithelial and vascular tissues and function as a linker protein providing attachment for cytoskeletal elements such as intermediate filaments (Cabral et al., Cell Tissue Res, 341:121-129, 2010). The third peptide (clone=P197-Bp4-830) was related to F-actin binding domain of Ber/Abl/cAb I (Hantschel et al., Mol Cell, 19:461-473, 2005). Two relatively small peptides had homology to C—C motif chemokine 22 (CCL22) and IL-17R. CCL22 is produced by tissue-resident macrophages and modulates Th1/Th2 responses (Ushio et al., Front Immunol, 9:2594, 2018). IL-17R is the receptor for IL-17 but also plays a role in to limit the signaling pathway via the internalization of its ligand, thereby it controls IL-17 pathway (Kurte et al., Front Immunol, 9:802, 2018). A mimotope with a relatively large sequence (39AA) with homology to response gene to complement 32 (RGC32) was identified. RGC32 is induced by p53 in response to DNA damage and expressed in various tissues and is involved in various physiological and pathological processes, including cell proliferation, differentiation, fibrosis, metabolic disease (Cui et al., Front Cardiovasc Med, 5:128, 2018). The corresponding gene is involved in angiogenesis is and regulated through hypoxia response element (An et al., Circulation, 120:617, 2009). A sequence with 17aa had homology to probable C-mannosyltransferase DPY19L2, which mediates the C-mannosylation of tryptophan residues on client proteins, including type I cytokine receptors (Niwa et al., Mol Biol Cell, 27:744-756, 2016). Two different clones (p51-BP4-457 and p51-BP3-57) with reduced expression in sarcoidosis had the same sequences with homology to POLB. POLB acts as a DNA polymerase is one of key enzymes for DNA repair (Sobol, PLoS Genet, 8:e1003086, 2012). Previously, autoantibody against POLB has been described in lupus erythematosus (Luo et al., Genomics Proteomics Bioinfor, 17:248-259, 2019). This was experimentally confirmed by mutation of POLB in mice that spontaneously developed lupus like syndrome (Senejani et al., Cell Rep, 6:1-8, 2014). Another clone had homology with INADL protein. INADL protein has multiple PDZ domains and interacts as scaffold protein to organize multimeric protein complexes at the cell membrane (Nourry et al, Sci STKE, 2003(179):RE7, 2003).

Because various drugs may affect the autoantibody production, in current study, immunoscreening was performed using a set of sera from sarcoidosis subjects with no prior treatment. In spite of this, several shared antigenic clones between non-treated subjects (current study) and a previous study were found, in which samples derived from subjects, who were partly treated with immunosuppressive medication. Sets of classifiers with different sensitivity and specificity were found. Some show increased expression and others showed decreased expression. Because sarcoidosis is a chronic disease involving many organs, the variation of autoantibodies expression profile may differ in early stages versus later stages or in various organ involvement. Although natural antibodies may also be beneficial to remove and neutralize pathogens, autoantibodies can directly interact with FCγ receptors or Toll-like receptors to initiate or amplify inflammation and perpetuate autoantibody production. Pathogenic autoantibodies can protect or cause diseases via neutralization of self-antigens, opsonization, antibody-dependent cellular cytotoxicity, activation of the complement system, pro-inflammatory and anti-inflammatory effect. Because of their broad reactivity for a wide variety of microbial components, natural antibodies have a major role in the primary line of defense against infections. Because some IgG autoantibodies may function as neutralization of pathogenic processes, the identification of decreased autoantibodies may be useful as therapeutics. Several studies, including this study indicate that in sarcoidosis FCγ receptors play a role in sarcoidosis (Talreja et al., Sci Rep. 7(1):2720, 2019). The identification of autoantibodies in sarcoidosis is important, as they may contribute to the cause of disease. However these autoantibodies need further experimental validation or confirmation using different avenue such as ELISA to elucidate their role in the detection of sarcoidosis or in organ involvement of this disease.

TABLE 7

| | | Peptide | | |
|---|---|---|---|---|
| | | sequence of | | |
| | | mimotopes | Description of the | |
| | Clone and | in-frame with | sequences that | |
| Rank | Peptide size | T7 10B gene | mimotopes mimic | Region of similarity of peptide |
| 1 | P197_BP4_922 | SACLQSLRTQLLT | cofilin 1 (non- | Id = 7/7 (100%) Gaps = 0/7 (0%) Length = 149 |
| | (23 aa) | WALVGDVGQP | muscle), isoform | Query 16 LVGDVGQ 22 |
| | | (SEQ ID NO: 1) | CRA_a [Homo sapiens] | LVGDVGQ |
| | | | Sequence ID: | Sbjct 39 LVGDVGQ 45 |
| | | | EAW74448.1 | LQSLRTQLLT |
| 2 | P197_BP4_921 | AGISRELVDKLAAA | Chain A. Human | Id = 11/11 (100%) Gaps = 0/11 (0%) Length = 326 |
| | (16 aa) | LE | Metap1 | Query 6 ELVDKLAAALE 16 |
| | | (SEQ ID NO: 2) | Sequence ID: 4FLI_A | ELVDKLAAALE |
| | | | | Sbjct 310 ELVDKLAAALE 320 |
| 3 | P197_BP4_923 | RKRRQ | inositol 1,4,5- | Id = 5/5(100% 0 Gaps = 0/5(0%) Length = 267 |
| | (5 aa) | (SEQ ID NO: 3) | trisphosphate | Query 1 RKRRQ 5 |
| | | | receptor type 3 | RKKRQ |
| | | | [Homo sapiens] | Sbjct 2654 RKRRQ 2658 |
| | | | Sequence ID: | |
| | | | NP_002215.2 | |
| 4 | P197_BP4_1112 | SDSCPHRP | C-C motif chemokine | Id = 7/8 Gaps = 1/8 (12%) Length = 93 |
| | (8 aa) | (SEQ ID NO: 4) | 22 precursor | Query 1 SDSCPHRP 8 |
| | | | [Homo sapiens] | SDSCP RP |
| | | | Sequence ID: | Sbjct 57 SDSCP-RP 63 |
| | | | NP_002981.2 | |
| 5 | P197_BP4_909 | SKNLYSPYTEASIE | Chain A. Desmoplakin | Id = 8/10(805%) Gaps = 0/10(0%) Length = 450 |
| | (21 aa) | LHLNSHS | [Homo sapiens] | Query 11 ASIELHLNSH 20 |
| | | (SEQ ID NO: 5) | Sequence ID: 3R8N_A | AS+E H NSH |
| | | | | Sbjct 35 ASVEQHINSH 44 |
| | | | | chondroitin sulfate N-acetylgalactosaminyl- |
| | | | | transferase 1-like isoform X2 |
| 6 | P197_BP4_930 | SSLGCCECKSVR | ras-related protein | Id = 6/6(100%) Gaps = 0/6(0%) Length = 357 |
| | (12 aa) | (SEQ ID NO: 6) | Rab-36 isoform 1 | Query 1 SSLGCC 6 |
| | | | [Homo sapiens] | SSLGCC |
| | | | Sequence ID: | Sbjct 352 SSLGCC 357 |
| | | | NP_001336806.1 | |
| 7 | P51_BP3_176 | SEKHPHRP | apoptosis related | Id = 6/6(100%) Gaps = 0/6(0%) Length = 114 |
| | (8 aa) | (SEQ ID NO: 7) | protein APR-4, | Query 2 EKHPHR 7 |
| | | | partial | +KHPHR |
| | | | [Homo sapiens] | Sbjct 59 QKHPHR 64 |
| | | | Sequence ID: | |
| | | | AAD31316.1 | |
| 8 | P51-BP4-523 | TDSTPALLSATVTP | Response gene to | Id = 39/39(100%) Gaps = 0/39(0%) Length = 78 |
| | (39 aa) | QKAKLGDTKELEAF | complement 32, | Query 1 TDSTPALLSATVTPQKAKLGDTKELE |
| | | IADLDKTLASM | isoform CRA_b | AFIADLDKTLASM 39 |
| | | (SEQ ID NO: 8) | [Homo sapiens] | TDSTPALLSATVTPQKAKLGDTKELE |
| | | | Sequence ID: | AFIADLDKTLASM |
| | | | EAX08664.1 | Sbjct 40 TDSTPALLSATVTPQKAKLGDTKELE |
| | | | | AFIADLDKTLASM 78 |
| 9 | P51-BP3-322 | SSERNGQFPWPLKM | probable C- | Id = 6/6(100%) Gaps = 0/6(0%) Length = 421 |
| | (17 aa) | FLT | mannosyltransferase | Query 12 LKMFLT 17 |
| | | (SEQ ID NO: 9) | DPY19L2 isoform X17 | LKMFLT |
| | | | [Homo sapiens] | Sbjct 219 LKMFLT 224 |
| | | | Sequence ID: | |
| | | | XP_011536520.1 | |
| 10 | P51_BP3_339 | KFFQNLS | receptor tyrosine- | Id = 6/6(100%) Gaps = 0/6(0%) Length = 1349 |
| | (7 aa) | (SEQ ID NO: 10) | protein kinase | Query 2 KFFQNL 7 |
| | | | erbB-4 isoform X1 | KFFQNL |
| | | | [Homo sapiens] | Sbjct 1043 KFFQNL 1048 |
| | | | Sequence ID: | |
| | | | XP_016859066.1 | |
| 11 | P51-BP3-361 | INTDSIKLIA | neurite extension | Id = 6/6 (100%) Gaps = 0/6 (0%) Length = 1516 |
| | (10 aa) | (SEQ ID NO: 11) | and migration factor | Query 2 NTDSIK 7 |
| | | | [Homo sapiens] | NTDSIK |
| | | | Sequence ID: | Sbjct 598 NTDSIK 603 |
| | | | NP_001008537.1 | 830 |
| 12 | P197-BP4-830 | SKNLYSFLY | Solution structure | Id = 6/6(100%) Gaps = 0/6(0%) Length = 130 |
| | (9 aa) | (SEQ ID NO: 12) | of the F-actin | Query 2 KNLYSF 7 |
| | | | binding domain of | KNLYSF |
| | | | Bcr-Abl/c-Abl | Sbjct 59 KNLYSF 64 |
| | | | [Homo sapiens] | |
| | | | Sequence ID: 1ZZP_A | |
| 13 | P51_BP3_129 | SVDCRTCC | Interleukin 17A | Id = 6/7(86%) Gaps = 1/7 (14%) Length = 155 |
| | (8 aa) | (SEQ ID NO: 13) | [Homo sapiens] | Query 1 SVDCRTC 7 |
| | | | Sequence ID: | SVDC TC |
| | | | AAH66253.1 | Sbjct 141 SVDC-TC 146 |
| 14 | P197_BP4_745 | SNEANRFSFILVLRG | SH3 domain-con- | Id = 45/47(95%) Gaps = 2/47(5%) Length = 246 |
| | (70 aa) | CYNFLFLWSLEGSCL | taining YSC84-like | Query 24 SLEGSCLIERKETNRKFYDIRAYDIL |
| | | IERKETNRKFYDIRA | protein 1 isoform | FGDTPRPAQAEDLYEILDS 70 |
| | | YDILFGDTPRPAQAE | 4 [Homo sapiens] | SLEGSCLIERKETNRKFYDIRAYDIL |
| | | DLYEILDSLY | Sequence ID: | FGDTPRPAQAEDLYEILDS |
| | | (SEQ ID NO: 14) | NP_001289616.1 | Sbjct 67 SLEGSCLIERKETNRKFYCQDIRAYDIL |
| | | | | FGDTPRPAQAEDLYEILDS 113 |
| 15 | P197_BP4_754 | DEIFTLKLIEGGALG | ras-related | Id = 9/10(90%) Gaps = 1/10(10%) Length = 244 |
| | (25 aa) | KCEVMRVEPS | protein Rab-12 | Query 1 DEIFTLKLIE 10 |
| | | (SEQ ID NO: 15) | [Homo sapiens] | DEIF LKL++ |
| | | | Sequence ID: | Sbict 194 DEIF-LKLVD 202 |
| | | | NP_001020471.2 | |
| 16 | P197_BP4_753 | KFFQNLS | receptor tyrosine- | Id = 6/6 (100%) Gaps = 0/6 (0%) Length = 1349 |
| | (7 aa) | (SEQ ID NO: 10) | protein kinase | Query 1 KFFQNL 6 |
| | | | erbB-4 isoform: X1 | KFFQNL |
| | | | [Homo sapiens] | Sbjct 1043 KFFQNL 1048 |
| | | | Sequence ID: | |
| | | | XP_016859066.1 | |
| 17 | P197_BP4_751 | SVAVSQDCTTALHPG | transformation- | Id = 18/24(75%) Gaps = 0/24(0%) Length = 56 |
| | (43 aa) | QQSETLSQKKKGLQR | related protein 10 | Query 2 VAVSQDCTTALHPGQQSETLSQKK 25 |
| | | XRQDYFFXLNLFF | [Homo sapiens] | VAVS+D AL PG QSET SQKK |
| | | (SEQ ID NO: 16) | Sequence ID: | Sbjct 27 VAVSRDRANALQPGLQSETFSQKK 50 |
| | | | AAQ18032.1 | |
| 18 | P51_BP3_57 | GKYNSTFTSSIIHNK | beta-polymerase | Id = 8/11 (73%) Gaps = 0/11(0%) Length = 335 |
| | (18 aa) | NMK | [Homo sapiens] | Query 7 FTSSIIHNKNM 17 |
| | | (SEQ ID NO: 17) | Sequence ID: | FT SI NKNM |
| | | | AAA60133.1 | Sbjct 272 FTGSDIFNKNM 282 |
| 19 | P51_BP4_475 | GKYNSTFTSSIIHNK | beta-polymerase | Id = 8/11 (73%) Gaps = 0/11(0%) Length = 335 |
| | (18 aa) | NMK | [Homo sapiens] | Query 7 FTSSIIHNKNM 17 |
| | | (SEQ ID NO: 17) | Sequence ID: | FT SI NKNM |
| | | | AAA60133.1 | Sbjct 272 FTGSDIFNKNM 282 |
| 20 | P51-BP3-34 | SGSLEVRSCTPAWVT | INADL protein | Id = 14/22 (64%) Gaps = 3/22 (13%) Length = 1181 |
| | (25 aa) | ERNFISKKKG | [Homo sapiens] | Query 3 SLEVRSCTPAWVTERNFISKKK 24 |
| | | (SEQ ID NO: 18) | Sequence ID: | SL S TPAWVTE + +SKKK |
| | | | AAI42662.1 | Sbjct 1158 SL-SSTPAWVTEQDSVSKKK 1176 |

Standard reference works setting forth the general principles of immunology include Abbas et al., Cellular and Molecular Immunology (6th Ed.), W.B. Saunders Co., Philadelphia, 2007; Janeway et al., Immunobiology. The Immune System in Health and Disease, 6th ed., Garland Publishing Co., New York, 2005; Delves et al. (eds.) Roitt's Essential Immunology (11th ed.) Wiley-Blackwell, 2006; Roitt et al., Immunology (7 th ed.) C.V. Mosby Co., St. Louis, Mo. (2006); Klein et al., Immunology (2nd ed), Blackwell Scientific Publications, Inc., Cambridge, Mass., (1997).

Additionally, methods particularly useful for polyclonal and monoclonal antibody production, isolation, characterization, and use are described in the following standard references: Harlow et al., Antibodies: A Laboratory Manual, Cold Spring Harbor Laboratory Press, Cold Spring Harbor, N.Y., 1988); Harlow et al., Using Antibodies: A Laboratory Manual, Cold Spring Harbor Laboratory Press, Cold Spring Harbor Laboratory Press, Cold Spring Harbor, N.Y., 1998; Monoclonal Antibodies and Hybridomas: A New Dimension in Biological Analyses, Plenum Press, New York, N.Y. (1980); Zola et al., in Monoclonal Hybridoma Antibodies: Techniques and Applications, CRC Press, 1982).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in the ability to diagnose a sarcoidosis subject from a healthy subject or a sarcoidosis subject from a tuberculosis subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. A method of diagnosing sarcoidosis in a subject comprising:
assaying a sample derived from a subject for the presence of one or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and
diagnosing the subject as healthy or having sarcoidosis based on the up- or down-regulation of the one or more markers, as compared to a reference level for each marker.

2. The method of claim 1 comprising one or more of:
assaying the sample for the presence of CFL1, 4FLI_A, ITPR3, CCL22, DSP, RAB36, PAR4, RGC32, DPY19L2, ERBB4, NEXMIF, or 1ZZP; and diagnosing the subject as healthy or having sarcoidosis based on the up- or down-regulation of the one or more markers; or
assaying the sample for the presence of IL17A, SH3YL1, RAB12, TRG10, POLKB, or INADL; and diagnosing the subject as healthy or having sarcoidosis based on the up- or down-regulation of the one or more markers; or
assaying the sample for the presence of two, three, four, five, six, seven, eight, nine, ten, eleven, twelve, thirteen, or more of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and diagnosing the subject as healthy or having sarcoidosis based on the up- or down-regulation of the one or more markers; or
assaying the sample for the presence of at least one of CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; and diagnosing the subject as healthy or having sarcoidosis based on the up- or down-regulation of the one or more markers.

3-5. (canceled)

6. A kit for diagnosing sarcoidosis in a subject wherein the kit comprises a protein that binds one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; and a detectable label.

7. The kit according to claim 6 comprising:
one or more proteins that bind one of CFL1, 4FLI_A, ITPR3, CCL22, DSP, RAB36, PAR4, RGC32, DPY19L2, ERBB4, NEXMIF, or 1ZZP; and a detectable label; or
one or more proteins that bind IL17A, SH3YL1, RAB12, TRG10, POLKB, or INADL; and
a detectable label; or
two, three, four, five, six, seven, eight, nine, ten, eleven, twelve, thirteen, or more proteins that each one of bind of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL, and a detectable label; or
one or more proteins that bind CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; and a detectable label.

8-10. (canceled)

11. The kit according to claim 6, wherein the proteins comprise antibodies, epitopes or mimotopes.

12. A kit for diagnosing sarcoidosis in a subject wherein the kit comprises a nucleic acid that binds a gene encoding CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; and a detectable label.

13. The kit according to claim 12 comprising:
one or more nucleic acids that bind a gene encoding CFL1, 4FLI_A, ITPR3, CCL22, DSP, RAB36, PAR4, RGC32, DPY19L2, ERBB4, NEXMIF, or 1ZZP; and a detectable label; or
one or more nucleic acids that bind a gene encoding IL17A, SH3YL1, RAB12, TRG10, POLKB, or INADL; and a detectable label; or
two, three, four, five, six, seven, eight, nine, ten, eleven, twelve, thirteen, or more nucleic acids each of which binds a gene encoding one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; and a detectable label; or
one or more nucleic acids that bind a gene encoding CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; and a detectable label.

14-16. (canceled)

17. The kit according to claim 6 wherein the detectable label is a radioactive isotope, enzyme, dye, fluorescent dye, magnetic bead, or biotin.

18. The kit according claim 6, further comprising reagents to perform an enzyme-linked immunosorbent assay (ELISA), a radioimmunoassay (RIA), a Western blot, an immunoprecipitation, an immunohistochemical staining, flow cytometry, fluorescence-activated cell sorting (FACS), an enzyme substrate color method, and/or an antigen-antibody agglutination.

19. A method of diagnosing sarcoidosis in a subject comprising:
obtaining a sample from a subject; assaying the sample for one or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL;
obtaining a value based on the assay; comparing the value to a reference level; and
diagnosing the subject as healthy or having sarcoidosis based on the up- or down-regulation of the one or more markers as demonstrated by the value and the reference level.

20. The method according to claim 19, comprising one or more of:
assaying the sample for one or more markers selected from CFL1, 4FLI_A, ITPR3, CCL22, DSP, RAB36, PAR4, RGC32, DPY19L2, ERBB4, NEXMIF, and 1ZZP; or
assaying the sample for one or more markers selected from IL17A, SH3YL1, RAB12, TRG10, POLKB, and INADL; or
assaying the sample for two, three, four, five, six, seven, eight, nine, ten, eleven, twelve, thirteen, or more markers selected from CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, and INADL; or
comprising assaying the sample for one or more markers selected from CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2.

21-23. (canceled)

24. The method according to claim 1, wherein one or more of:
assaying the sample for one or more markers comprise contacting the sample with a probe comprising a detectable label, wherein the probe binds the marker;
obtaining a value based on the assay comprises analyzing the binding of the probe to the marker in the sample;
analyzing the binding of the probe to the marker in the sample comprises quantitating the amount of the marker in the sample;
the sample is a tissue sample, a cell sample, a whole blood sample, a serum sample, a plasma sample, a saliva sample, a sputum sample, or a urine sample;
the value is a score;
the value is a weighted score.

25-29. (canceled)

30. A microarray comprising:
one or more proteins each of which binds one of CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; or
one or more proteins each of which binds one of CFL1, 4FLI_A, ITPR3, CCL22, DSP, RAB36, PAR4, RGC32, DPY19L2, ERBB4, NEXMIF, or 1ZZP; or
one or more proteins each of which binds one of IL17A, SH3YL1, RAB12, TRG10, POLKB, or INADL; or
a nucleic acid that binds to a gene encoding CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL; or
a nucleic acid that binds a gene encoding CFL1, 4FLI_A, ITPR3, CCL22, DSP, RAB36, PAR4, RGC32, DPY19L2, ERBB4, NEXMIF, or 1ZZP; or
a nucleic acid that binds a gene encoding: IL17A, SH3YL1, RAB12, TRG10, POLKB, or INADL;
one or more of the following proteins or a identifying peptide therefrom CFL1, 4FLI_A, ITPR3, CCL22, DSP, RAB36, PAR4, RGC32, DPY19L2, ERBB4, NEXMIF, 1ZZP, IL17A, SH3YL1, RAB12, TRG10, POLKB, or INADL;
one or more of the following proteins or a identifying peptide therefrom CFL1, 4FLI_A, ITPR3, CCL22, DSP, RAB36, PAR4, RGC32, DPY19L2, ERBB4, NEXMIF, or 1ZZP;
one or more of the following proteins or a identifying peptide therefrom: IL17A, SH3YL1, RAB12, TRG10, POLKB, or INADL.

31-32. (canceled)

33. The microarray of claim 30, further comprising:
one or more proteins each of which binds one of CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; or
at least one nucleic acid that binds a gene encoding CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2; or
one or more of the following proteins or a identifying peptide therefrom: CCL21; Metap1; PC4; CLI_3190; TNFRSF21; CD14; DNAJC1; APBB1; FGFBP-2; SH3YL1 Fed A; WDFY3; MFS; LRPPRC; HLA-DR; TKT; Rv0189C; BfrA; DAB2; or TCEB2.

34-41. (canceled)

42. The microarray of claim 30, wherein one or more of:
the protein or the nucleic acid on the microarray comprises a label that can be detected; or
the microarray comprises two or more, three or more, four or more, five or more, six or more, seven or more, eight or more, or nine or more of the proteins on the microarray; or
comprises two or more, three or more, four or more, five or more, six or more, seven or more, eight or more, or nine or more of the nucleic acids on the microarray.

43-44. (canceled)

45. A kit comprising the microarray of claim 30.

46. A kit according to claim 6, wherein the kit utilizes at least one clone or marker sequence identified herein, and wherein the kit comprises reagents to perform an enzyme-linked immunosorbent assay (ELISA), to detect specific immunoglobulin (IgG, IgA and Ig M).

47. A method of serological diagnosis of sarcoidosis, and/or a method of distinguishing sarcoidosis from other granulomatous diseases (such as tuberculosis), comprising detecting one or more immunoglobulin (IgG, IgA and Ig M) specific for and/or immunoreactive to at least one clone or marker sequence identified herein.
